SEQUENCE LISTING (10 sequences)

SEQ ID NO: 1 - protein, 121 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 5 aa, synthetic construct
SSWMN

SEQ ID NO: 3 - protein, 17 aa, synthetic construct
RIYPGDGDTNYNVKFKG

SEQ ID NO: 4 - protein, 12 aa, synthetic construct
SGFITTVRDFDY

SEQ ID NO: 5 - protein, 111 aa, synthetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 6 - protein, 15 aa, synthetic construct
RASESVDTFGISFMN

SEQ ID NO: 7 - protein, 7 aa, synthetic construct
EASNQGS

SEQ ID NO: 8 - protein, 9 aa, synthetic construct
QQSKEVPFT

SEQ ID NO: 9 - protein, 451 aa, synthetic construct
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0217823 A1
(43) Pub. Date: Jul. 30, 2026

(54) ANTI-CD19 BINDING AGENTS FOR THE TREATMENT AND PREVENTION OF IMMUNE MEDIATED NECROTIZING MYOPATHY

(71) Applicant: Viela Bio, Inc., Deerfield, IL (US)

(72) Inventors: Nishi RAMPAL, Branford, CT (US); Yanping WU, Potomac, MD (US)

(73) Assignee: Viela Bio, Inc., Deerfield, IL (US)

(21) Appl. No.: 19/148,817

(22) PCT Filed: Jan. 18, 2024
(86) PCT No.: PCT/US2024/012011

§ 371 Date: Jul. 17, 2025

Related U.S. Application Data
(60) Provisional application No. 63/480,908, filed on Jan. 20, 2023.

(51) Int. Cl. C07K 16/28; A61K 45/06; A61P 37/06

(52) U.S. Cl. CPC C07K 16/2803; A61K 45/06; A61P 37/06

(57) ABSTRACT

The present disclosure is related to compositions and method of treating disease (e.g., myositis) in a subject in need thereof using anti-CD19 antibodies or antigen-binding fragments thereof.

### CROSS REFERENCE TO RELATED APPLICATIONS

The present application claims priority to U.S. Provisional Patent Application No. 63/480,908, filed Jan. 20, 2023, which is incorporated by reference herein in its entirety for all purposes.

### SEQUENCE LISTING

The contents of the electronic sequence listing (HOPA_064_01WO_SeqList_ST26.xml; Size: 10,639 bytes; and Date of Creation: Jan. 4, 2024) are herein incorporated by reference in its entirety.

### TECHNICAL FIELD

The present disclosure is related to compositions comprising anti-CD19 binding proteins and methods of using the same in the treatment and prevention of myopathies.

### BACKGROUND

Idiopathic inflammatory myopathies can result in permanent muscle damage with weakness if not controlled promptly. Currently, corticosteroids, intravenous immunoglobulin (IVIG), and other immune modulators are used for treatment, but these medications are also associated with side effects including risk for diabetes, osteoporosis, cataracts, opportunistic infections, and thrombosis. In addition, some subjects do not achieve timely control of their disease with these therapies. This, together with the burden of their side effects, yields the clinical need for a therapy to treat these patients.

Cluster of differentiation 19 (CD19) can be targeted for treatment and prevention of myopathies. Therefore, provided are compositions and methods of utilizing anti-CD19 antibodies or antigen-binding fragments thereof to reduce or eliminate symptoms or diseases associated with CD19.

### BRIEF SUMMARY

Provided are methods of treating Immune Mediated Necrotizing Myopathy (IMNM), the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8.

Provided are methods of treating Immune Mediated Necrotizing Myopathy (IMNM), the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, and wherein the subject in need thereof is pediatric. In aspects, the pediatric subject is 16-18 years old.

Provided are methods of treating Immune Mediated Necrotizing Myopathy (IMNM), the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is from about 100 mg-1000 mg.

Provided are methods of treating Immune Mediated Necrotizing Myopathy (IMNM), the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is about 300 mg.

Provided are methods of treatment, the methods comprising administering an effective amount of an anti-CD19 antibody or antigen-binding fragment thereof to a pediatric subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively.

Provided are methods of treating Immune Mediated Necrotizing Myopathy (IMNM), the methods comprising administering an effective amount of inebilizumab to a subject in need thereof, wherein the effective amount is about 100 mg-1000 mg.

Provided are methods of treatment, the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is positive for autoantibodies selected from the group consisting of: signal recognition particle (SRP), 3-hydroxy-3-methylglutaryl-coenzyme A reductase (HMGCR), and both SRP and HMGCR.

Provided are methods of treatment, the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is negative for autoantibodies selected from the group consisting of: signal recognition particle (SRP), 3-hydroxy-3-methylglutaryl-coenzyme A reductase (HMGCR), and both SRP and HMGCR.

In aspects, the subject is signal recognition particle-antibody positive (SRP-Ab+), 3-hydroxy-3-methylglutaryl-coenzyme A reductase-antibody positive (HMGCR-Ab+), or both SRP-Ab+ and HMGCR-Ab+. In aspects, the subject is SRP-Ab+. In aspects, the subject is HMGCR-Ab+. In aspects, the subject is signal recognition particle-antibody negative (SRP-Ab−), 3-hydroxy-3-methylglutaryl-coenzyme A reductase-antibody negative (HMGCR-Ab−), or both SRP-Ab− and HMGCR-Ab−. In aspects, the subject in need was previously treated with a conventional therapy. In aspects, the conventional therapy comprises a corticosteroid, a non-steroidal immunosuppressant (IST), antibody therapy, or any combination thereof. In aspects, the conventional therapy is a corticosteroid, and wherein the corticosteroid is prednisone. In aspects, the conventional therapy is an IST selected from the group consisting of: methotrexate, cyclophosphamide, cyclosporine, azathioprine, mycophenolate mofetil, mycophenolic acid, tacrolimus, and any combination thereof. In aspects, the conventional therapy is an antibody therapy is selected from the group consisting of: rituximab, ocrelizumab, obinutuzumab, ofatumumab, inebilizumab intravenous immunoglobulin (IVIg), and combinations thereof. In aspects, the conventional therapy is tapered following the administering of the effective amount of the anti-CD19 antibody or antigen-binding fragment thereof. In aspects, the tapering comprises reducing a dosage of the conventional therapy every two weeks beginning on week 26. In aspects, the conventional therapy comprises prednisone, and wherein the prednisone is tapered by 5 mg every two weeks following the administering. In aspects, the tapering is continued at least until week 12. In aspects, the subject was determined to have at least one of SRP-Ab or HMGCR-Ab before the administering. In aspects, the administering of the anti-CD19 antibody or antigen-binding fragment thereof is repeated. In aspects, the administering is repeated on weeks 2, 26, 52, 78, 104, 130, and 156. In aspects, the administering, the repeated administering, or both are effective at reducing disease or symptoms thereof by week 12, 26, or 52. In aspects, the administering, the repeated administering, or both are effective at reducing disease or symptoms thereof by week 26 following the administering as determined by an increased 2016 ACR/EULAR total improvement score (TIS) as compared to a baseline score of the subject or a score in an untreated subject. In aspects, the administering, the repeated administering, or both are effective at reducing disease or symptoms thereof by week 26 following the administering as compared to a baseline level of the subject or a level in an untreated subject. In aspects, the reduced disease or symptoms thereof are determined by an improvement in muscle weakness, reduction in serum CK level, reduced atrophy, reduced edema, reduced fatty tissue replacement, reduced interstitial lung disease, reduced B cell levels, MMT8 score, and combinations thereof. In aspects, the subject is human. In aspects, the disease reduction is of at least about 3-fold, 5-fold, 20-fold, 40-fold, 60-fold, 80-fold, or up to about 100-fold. In aspects, the TIS score is ≥5, 10, 20, 30, 40, 50, 60, 80, or 100. In aspects, before the administering the subject in need thereof is prophylactically administered a glucocorticoid, an antihistamine, an anti-pain agent, and any combination thereof. In aspects, the subject is prophylactically administered the glucocorticoid, the antihistamine, and the anti-pain agent. In aspects, the subject is prophylactically administered methylprednisolone, diphenhydramine, and acetaminophen. In aspects, the effective amount of the anti-CD19 antibody or antigen-binding fragment thereof is from about: 50 mg to about 1000 mg, 100-800 mg, 200-600 mg, 200-500 mg, 250-450 mg, 200-350 mg, 250-350 mg, or 375-325 mg. In aspects, the effective amount is from about: 50 mg, 100 mg, 200 mg, 225 mg, 250 mg, 275 mg, 300 mg, 325 mg, 350 mg, 375 mg, 400 mg, 500 mg, 600 mg, 700 mg, 800 mg, 900 mg, or 1000 mg. In aspects, the effective amount is about 300 mg. In aspects, the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively. In aspects, the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively. In aspects, the anti-CD19 antibody is a monoclonal antibody. In aspects, the anti-CD19 antibody is afucosylated. In aspects, the administering, the repeated administering, or both are effective at eliminating the IMNM for at least about 3 months, 6 months, 1 year, or 2 years. In aspects, the administering, the repeated administering, or both are effective at stabilizing or reducing the IMNM for at least about 3 months, 6 months, 1 year, or 2 years.

In aspects, the administering of any of the methods of the disclosure comprises 300 mg of the anti-CD19 antibody or antigen-binding fragment thereof on day 1 and day 15, followed by a single 300 mg infusion approximately every 6 months thereafter.

Provided are also methods of treating Immune Mediated Necrotizing Myopathy (IMNM), the methods comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is about 300 mg and is administered every 6 months, and wherein two weeks prior to the first 300 mg administration every 6 months, an initial 300 mg dose is administered. In aspects, the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively. In aspects, the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.

Provided are anti-CD19 antibodies or antigen-binding fragment thereof for use in the treatment of Immune Mediated Necrotizing Myopathy (IMNM), the anti-CD19 antibody or antigen-binding fragment thereof, comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8. Provided are also anti-CD19 antibodies or antigen-binding fragment thereof for use in the treatment of Immune Mediated Necrotizing Myopathy (IMNM) in a pediatric subject, the anti-CD19 antibody or antigen-binding fragment thereof, comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8. In aspects, the pediatric subject is 16-18 years old. In aspects, the subject is signal recognition particle-antibody positive (SRP-Ab+), 3-hydroxy-3-methylglutaryl-coenzyme A reductase-antibody positive (HMGCR-Ab+), or both SRP-Ab+ and HMGCR-Ab+. In aspects, the subject is SRP-Ab+. In aspects, the subject is HMGCR-Ab+. In aspects, the subject is signal recognition particle-antibody negative (SRP-Ab−), 3-hydroxy-3-methylglutaryl-coenzyme A reductase-antibody negative (HMGCR-Ab−), or both SRP-Ab− and HMGCR-Ab−. In aspects, the subject in need was previously treated with a conventional therapy. In aspects, the conventional therapy comprises a corticosteroid, a non-steroidal immunosuppressant (IST), antibody therapy, or any combination thereof. In aspects, the conventional therapy is a corticosteroid, and the corticosteroid is prednisone. In aspects, the conventional therapy is an IST selected from the group consisting of: methotrexate, cyclophosphamide, cyclosporine, azathioprine, mycophenolate mofetil, mycophenolic acid, tacrolimus, and any combination thereof. In aspects, the conventional therapy is an antibody therapy is selected from the group consisting of: rituximab, ocrelizumab, obinutuzumab, ofatumumab, inebilizumab intravenous immunoglobulin (IVIg), and combinations thereof. In aspects, the conventional therapy is tapered following the administering of the effective amount of the anti-CD19 antibody or antigen-binding fragment thereof. In aspects, the tapering comprises reducing a dosage of the conventional therapy every two weeks beginning on week 26. In aspects, the conventional therapy comprises prednisone, and the prednisone is tapered by 5 mg every two weeks following the administering. In aspects, the tapering is continued at least until week 12. In aspects, the subject was determined to have at least one of SRP-Ab or HMGCR-Ab before the administering. In aspects, the administering of the anti-CD19 antibody or antigen-binding fragment thereof is repeated. In aspects, the administering is repeated on weeks 2, 26, 52, 78, 104, 130, and 156. In aspects, the administering, the repeated administering, or both are effective at reducing disease or symptoms thereof by week 12, 26, or 52. In aspects, the administering, the repeated administering, or both are effective at reducing disease or symptoms thereof by week 26 following the administering as determined by an increased 2016 ACR/EULAR total improvement score (TIS) as compared to a baseline score of the subject or a score in an untreated subject. In aspects, the administering, the repeated administering, or both are effective at reducing disease or symptoms thereof by week 26 following the administering as compared to a baseline level of the subject or a level in an untreated subject. In aspects, the reduced disease or symptoms thereof are determined by an improvement in muscle weakness, reduction in serum CK level, reduced atrophy, reduced edema, reduced fatty tissue replacement, reduced interstitial lung disease, reduced B cell levels, MMT8 score, and combinations thereof. In aspects, the subject is human. In aspects, the disease reduction is of at least about 3-fold, 5-fold, 20-fold, 40-fold, 60-fold, 80-fold, or up to about 100-fold. In aspects, the TIS score is ≥5, 10, 20, 30, 40, 50, 60, 80, or 100. In aspects, before the administering the subject in need thereof is prophylactically administered a glucocorticoid, an antihistamine, an anti-pain agent, and any combination thereof. In aspects, the subject is prophylactically administered the glucocorticoid, the antihistamine, and the anti-pain agent. In aspects, the subject is prophylactically administered methylprednisolone, diphenhydramine, and acetaminophen. In aspects, the effective amount of the anti-CD19 antibody or antigen-binding fragment thereof is from about: 50 mg to about 1000 mg, 100-800 mg, 200-600 mg, 200-500 mg, 250-450 mg, 200-350 mg, 250-350 mg, or 375-325 mg. In aspects, the effective amount is from about: 50 mg, 100 mg, 200 mg, 225 mg, 250 mg, 275 mg, 300 mg, 325 mg, 350 mg, 375 mg, 400 mg, 500 mg, 600 mg, 700 mg, 800 mg, 900 mg, or 1000 mg. In aspects, the effective amount is about 300 mg. In aspects, the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively. In aspects, the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively. In aspects, the anti-CD19 antibody is a monoclonal antibody. In aspects, the anti-CD19 antibody is afucosylated. In aspects, the administering, the repeated administering, or both are effective at eliminating the IMNM for at least about 3 months, 6 months, 1 year, or 2 years. In aspects, the administering, the repeated administering, or both are effective at stabilizing or reducing the IMNM for at least about 3 months, 6 months, 1 year, or 2 years.

Provided are also anti-CD19 antibodies or antigen-binding fragment thereof for use in the treatment of Immune Mediated Necrotizing Myopathy (IMNM), the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein from about 100 mg-1000 mg of the anti-CD19 antibody or antigen-binding fragment thereof is administered to a subject in need. Provided are also anti-CD19 antibodies or antigen-binding fragment thereof for use in the treatment of Immune Mediated Necrotizing Myopathy (IMNM), the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein about 300 mg of the anti-CD19 antibody or antigen-binding fragment thereof is administered to a subject in need. Provided are also anti-CD19 antibodies or antigen-binding fragment thereof for use in the treatment of a disease associated with CD19 expressing cells in a pediatric subject, the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively. Provided is also Inebilizumab for use in the treatment of Immune Mediated Necrotizing Myopathy (IMNM), wherein from about 100 mg-1000 mg of the inebilizumab is administered to a subject in need. Provided are also anti-CD19 antibodies or antigen-binding fragment thereof for use in the treatment of a disease associated with CD19 expressing cells, the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is positive for autoantibodies selected from the group consisting of: signal recognition particle (SRP), 3-hydroxy-3-methylglutaryl-coenzyme A reductase (HMGCR), and both SRP and HMGCR. Provided are also anti-CD19 antibodies or antigen-binding fragment thereof for use in the treatment of a disease associated with CD19 expressing cells, the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is negative for autoantibodies selected from the group consisting of: signal recognition particle (SRP), 3-hydroxy-3-methylglutaryl-coenzyme A reductase (HMGCR), and both SRP and HMGCR. In aspects, the administering comprises 300 mg of the anti-CD19 antibody or antigen-binding fragment thereof on day 1 and day 15, followed by a single 300 mg infusion approximately every 6 months thereafter. Provided are also anti-CD19 antibodies or antigen-binding fragment thereof for use in the treatment of Immune Mediated Necrotizing Myopathy (IMNM), the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein about 300 mg is administered every 6 months, and wherein two weeks prior to the first 300 mg administration every 6 months, an initial 300 mg dose is administered. In aspects, the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively. In aspects, the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.

These and other aspects are described below.

### BRIEF DESCRIPTION OF FIGURES

The accompanying figures, which are incorporated herein and form a part of the specification, illustrate some, but not the only or exclusive, example aspects and/or features. It is intended that the aspects and figures disclosed herein are to be considered illustrative rather than limiting.

FIG. 1 depicts an exemplary study flow diagram for a trial where the randomized control period is 26 weeks.

FIG. 2 depicts an exemplary study flow diagram for a trial where the randomized control period is 52 weeks.

### DETAILED DESCRIPTION

The present disclosure provides antibodies and antigen-binding fragments thereof which bind to Cluster of Differentiation 19 (CD19, also known as B-lymphocyte antigen CD19). Inhibition of binding can reduce or eliminate activation of inflammatory signaling pathways associated with related disease. In aspects, anti-CD19 antibodies and antigen-binding fragments disclosed herein can be used to neutralize CD19. In aspects, anti-CD19 antibodies and antigen-binding fragments thereof disclosed herein can be used to regulate B cell activity. Provided herein are polypeptides comprising anti-CD19 antibodies and antigen-binding fragments thereof. Disclosed herein are methods of using the anti-CD19 antibodies and antigen-binding fragments thereof to improve myopathy-related quality of life. In aspects, the anti-CD19 antibodies and antigen-binding fragments thereof disclosed herein can be used to treat myopathies. In aspects, the anti-CD19 antibodies and antigen-binding fragments thereof disclosed herein can be used to treat immune-mediate necrotizing myopathy (IMNM).

### Definitions

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “subjects” are used interchangeably herein. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like. A subject can be pediatric.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, “treating” or “treat” describes the management and care of a subject for the purpose of combating a disease, condition, or disorder and includes the administration of an anti-CD19 antibody or antigen-binding fragment thereof used in the methods described herein to alleviate the symptoms or complications of a disease, condition, or disorder, or to eliminate the disease, condition, or disorder. Thus, the term “treat” or “treating” refers to both therapeutic measures and prophylactic or preventative measures, wherein the objective is to prevent, slow down (lessen), or ameliorate the progression of a disease (e.g., myositis or myopathy). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total).

When referring to a protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

### Cluster of Differentiation 19 (CD19) Binding Proteins

In aspects, provided herein are anti-CD19 binding agents. Anti-CD19 binding agents may be used in disclosed therapeutic regimens for the treatment of myositis (e.g., IMNM). IMNM is a debilitating and rapidly progressing form of idiopathic inflammatory myopathy with major impacts on a patient's muscle strength and functional activity. IMNM has been identified as an important and distinct disease with specific serological, clinical, and pathological diagnostic criteria (Allenbach et al, 2018). The hallmark features of IMNM are proximal muscle weakness, often the only clinical manifestation, and elevated creatine kinase (CK) levels. IMNM presents as 3 distinct subtypes depending on the status of auto-antibodies: Signal recognition particle-antibody positive (SRP-Ab+), 3-hydroxy-3-methylglutaryl-coenzyme A reductase-antibody positive (HMGCR-Ab+), or negative for both SRP-Ab and HMGCR-Ab (seronegative).

In aspects, a therapeutic regimen comprising any of the disclosed anti-CD19 binding agents results in B-cell reduction or elimination in a subject in need thereof. B cells, particularly plasmablasts and plasma cells, contribute to disease thereby reducing or eliminating them is of benefit to subjects with myositis. In aspects, an anti-CD19 binding agent is effective in reducing or eliminating at least one of CD19 positive: B cells, plasmablasts, plasma cells, or any combination thereof. In aspects, an anti-CD19 binding agent comprises an anti-CD19 antibody.

Exemplary sequences for anti-CD19 antibodies and antigen-binding fragments thereof are shown in Table 1. In aspects, an anti-CD19 antibody or antigen-binding fragment thereof comprises at least or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 10 shown in Table 1. In aspects, any one of the sequences from Table 1 can be modified. In aspects, a modification comprises one or more truncations, deletions, insertions, and combinations thereof. A modification can occur at any of the residues provided in Table 1 and in any number of residues from Table 1. In aspects, a modification can comprise from 1-3, 1-5, 1-10, 1-20, 3-8, 3-10, 3-15, 5-8, 5-10, or 5-20 residues. In aspects, a modification can occur in up to 1, 2, 3, 4, 5, 6, 7, 8, 9, 10, 11, 12, 13, 14, 15, 20, 30, 40, 50, 60, 70, 80, 90, 100, 200, 300, 400 or up to about 450 residues.

TABLE 1
sequences for a CD19 binding protein

| SEQ ID | | |
|---|---|---|
| NO | Description | Sequence |
| 1 | Variable Heavy (VH) | EVQLVESGGGLVQPGGSLRLSCAASGFTFSSSWMN |
| | | WVRQAPGKGLEWVGRIYPGDGDTNYNVKFKGRFT |
| | | ISRDDSKNSLYLQMNSLKTEDTAVYYCARSGFITTV |
| | | RDFDYWGQGTLVTVSS |
| 2 | VH CDR1 | SSWMN |
| 3 | VH CDR2 | RIYPGDGDTNYNVKFKG |
| 4 | VH CDR3 | SGFITTVRDFDY |
| 5 | Variable Light (VL) | EIVLTQSPDFQSVTPKEKVTITCRASESVDTFGISFM |
| | | NWFQQKPDQSPKLLIHEASNQGSGVPSRFSGSGSGT |
| | | DFTLTINSLEAEDAATYYCQQSKEVPFTFGGGTKVE |
| | | IK |
| 6 | VL CDR1 | RASESVDTFGISFMN |
| 7 | VL CDR2 | EASNQGS |
| 8 | VL CDR3 | QQSKEVPFT |
| 9 | Heavy Chain | EVQLVESGGGLVQPGGSLRLSCAASGFTFSSSWMN |
| | | WVRQAPGKGLEWVGRIYPGDGDTNYNVKFKGRFT |
| | | ISRDDSKNSLYLQMNSLKTEDTAVYYCARSGFITTV |
| | | RDFDYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKRVEPKSCDKTHTCPPCPAPELLGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 10 | Light Chain | EIVLTQSPDFQSVTPKEKVTITCRASESVDTFGISFM |
| | | NWFQQKPDQSPKLLIHEASNQGSGVPSRFSGSGSGT |
| | | DFTLTINSLEAEDAATYYCQQSKEVPFTFGGGTKVE |
| | | IKRTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSFNRG |
| | | EC |

In aspects, an anti-CD19 antibody or antigen-binding fragment thereof of the disclosure can comprise variants of the sequences provided they retain the ability to bind CD19. For example, conservative amino acid substitutions can be made within any one of the disclosed CDRs. Various alterations can be made according to the methods described in Antibody Engineering, 2nd ed., Oxford University Press, ed. Borrebaeck, 1995. These alterations comprise but are not limited to nucleotide sequences that are altered by the substitution of different codons that encode a functionally equivalent amino acid residue within the sequence, thus producing a “silent” change. For example, the nonpolar amino acids comprise alanine (A), leucine (L), isoleucine (I), valine (V), proline (P), phenylalanine (F), tryptophan (W), and methionine (M). The polar neutral amino acids comprise glycine (G), serine(S), threonine (T), cysteine (C), tyrosine (Y), asparagine (N), and glutamine (Q). The positively charged (basic) amino acids comprise arginine (R), lysine (K), and histidine (H). The negatively charged (acidic) amino acids comprise aspartic acid (D) and glutamic acid (E). In aspects, the CDR amino acid sequences disclosed herein (SEQ ID NOs: 2-4, 6-8) can vary by at least about 80%, 85%, 90%, 95%, 96%, 97%, 98%, 99%, or up to about 100%.

Derivatives and analogs of antibodies or antigen-binding fragments thereof of the disclosure can be produced by various techniques, including recombinant and synthetic methods (Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany). In aspects, a method for making a VH domain which is an amino acid sequence variant of a VH domain of the disclosure comprises a step of adding, deleting, substituting, or inserting one or more amino acids in the amino acid sequence of the presently disclosed VH domain, optionally combining the VH domain thus provided with one or more VL domains, and testing the VH domain or VH/VL combination or combinations for specific binding to the antigen. An analogous method can be employed in which one or more sequence variants of a VL domain disclosed herein are combined with one or more VH domains. In aspects, the amino acid VH or VL sequence disclosed herein (SEQ ID NOs: 1 and 5) can vary by at least about 80%, 85%, 90%, 95%, 96%, 97%, 98%, 99%, or up to about 100%.

The present disclosure provides methods for the use of CD19 binding molecules, e.g., antibodies and antigen-binding fragments thereof which bind CD19. The full-length amino acid and nucleotide sequences for CD19 are known in the art (see, e.g., UniProt Acc. No. Q71UW0 for human CD19). In aspects, an anti-CD19 antibody is also known as MEDI-551. In aspects, an anti-CD19 antibody is also known as VIB551. In aspects, an anti-CD19 antibody is also known as HZN551. In aspects, an anti-CD19 antibody is also known as UPLIZNA™. In aspects, an anti-CD19 antibody is also known as Inebilizumab. In aspects, compositions of the disclosure may comprise any of the amino acid sequences as described in International PCT Patent Application No. PCT/US2007/077916, which are incorporated herein by reference in their entireties (PCT/US2007/077916 refers to VIB551 as “16C4”). In aspects, provided compositions may comprise any of the sequences as shown in Table 1.

In aspects, an anti-CD19 antibody or antigen-binding fragment thereof disclosed herein can be glycosylated. In aspects, an anti-CD19 antibody or antigen-binding fragment thereof can have a glycosylation pattern that differs from the wild-type pattern. For example, one or more carbohydrate moieties can be deleted and/or one or more glycosylation sites added. Addition of glycosylation sites to the presently disclosed antibodies or antigen-binding fragments can be accomplished by altering the amino acid sequence to contain glycosylation site consensus sequences known in the art. Another means of increasing the number of carbohydrate moieties on the antibody or antigen-binding fragment is by chemical or enzymatic coupling of glycosides to the amino acid residues of the antibody or antigen-binding fragment. Such methods are described in WO 87/05330, and in Aplin et al. (1981) CRC Crit. Rev. Biochem., 22:259-306. Removal of any carbohydrate moieties from the antibodies or antigen-binding fragments may be accomplished chemically or enzymatically, for example, as described by Hakimuddin et al. (1987) Arch. Biochem. Biophys., 259:52; and Edge et al. (1981) Anal. Biochem., 118:131 and by Thotakura et al. (1987) Meth. Enzymol., 138:350.

In aspects, an anti-CD19 antibody or antigen-binding fragment thereof disclosed herein can be afucosylated. In aspects, an anti-CD19 antibody or antigen-binding fragment thereof can have a fucosylation pattern that differs from the wild-type pattern. For example, one or more fucose moieties can be deleted. Deletion of fucosylation sites to the presently disclosed antibodies or antigen-binding fragments can be accomplished by altering the amino acid sequence to remove fucosylation site consensus sequences known in the art. Another means of removal of any fucose moieties from the antibodies or antigen-binding fragments may be accomplished chemically or enzymatically, for example, as described by Hakimuddin et al. (1987) Arch. Biochem. Biophys., 259:52; and Edge et al. (1981) Anal. Biochem., 118:131 and by Thotakura et al. (1987) Meth. Enzymol., 138:350.

In aspects, an anti-CD19 binding agent comprises Inebilizumab. Inebilizumab is a humanized, affinity-optimized, afucosylated IgG1 kappa monoclonal antibody (mAb) known as 16C-aFuc that binds to the B-cell specific surface antigen CD19, resulting in reduction or elimination of CD19+ B cells. Inebilizumab is glycoengineered by expression of mAb 16C4 in a fucosyltransferase deficient Chinese hamster ovary producer cell line (BioWa Potelligent® Technology), which generates a homogenously afucosylated antibody with enhanced antibody-dependent cellular cytotoxicity (ADCC). In aspects, an anti-CD19 antibody or antigen-binding fragment thereof described herein mediates ADCC, complement-dependent cell-mediated cytotoxicity (CDC), and/or apoptosis. In aspects, an anti-CD19 antibody or antigen-binding fragment thereof described herein inhibits anti-IgM/CpG stimulated B cell proliferation.

### Dosing

In aspects, any of the compositions of the disclosure comprising an anti-CD19 antibody or antigen-binding fragment thereof and/or any of the conventional therapies disclosed herein can be administered in any form. In aspects, an anti-CD19 antibody or antigen-binding fragment thereof and/or any of the conventional therapies is administered intravenously, subcutaneously, orally, intramuscularly, intrathecally, sublingually, rectally, vaginally, cutaneously, systemically, topically, transdermally, or by way of inhalation. In aspects, an anti-CD19 antibody or antigen-binding fragment thereof is administered intravenously. In aspects, an anti-CD19 antibody or antigen-binding fragment thereof is administered by infusion.

An anti-CD19 antibody or antigen-binding fragment thereof of the disclosure can be administered at any dose. In aspects, an anti-CD19 antibody or antigen-binding fragment thereof is administered at a dose from about: 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, or about 1500 mg.

In aspects, an anti-CD19 antibody or antigen-binding fragment thereof is administered at a dose of between about: 50-100 mg, 50-150 mg, 100-200 mg, 130-180 mg, 150-250 mg, 200-300 mg, 250-350 mg, 50-1500 mg, 75-1200 mg, 100-800 mg, or 150-500 mg. In aspects, an anti-CD19 antibody or antigen-binding fragment thereof is administered at a dose selected from the group consisting of: 150 mg, 175 mg, 200 mg, 225 mg, 250 mg, 275 mg, 300 mg, 325 mg, 350 mg, 375 mg, 400 mg, 425 mg, 450 mg, 500 mg, 525 mg, 550 mg, 575 mg, and 600 mg. In aspects, an anti-CD19 antibody or antigen-binding fragment thereof is administered at an effective dose of about 300 mg. In aspects, 300 mg of an anti-CD19 antibody or antigen-binding fragment thereof is administered. In aspects, 300 mg of inebilizumab is administered.

Provided are also methods comprising administering conventional therapies.

In aspects, a conventional therapy can be administered from at least about or at most about: 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 6 mg/kg, 8 mg/kg, 10 mg/kg, 12 mg/kg, 14 mg/kg, 15 mg/kg, 16 mg/kg, 18 mg/kg, 20 mg/kg, 22 mg/kg, 24 mg/kg, 26 mg/kg, 28 mg/kg, 30 mg/kg, 32 mg/kg, 34 mg/kg, 36 mg/kg, 38 mg/kg, 40 mg/kg, 42 mg/kg, 44 mg/kg, 46 mg/kg, 48 mg/kg, 50 mg/kg, 52 mg/kg, 54 mg/kg, 56 mg/kg, 58 mg/kg, 60 mg/kg, 62 mg/kg, 64 mg/kg, 66 mg/kg, 68 mg/kg, 70 mg/kg, 72 mg/kg, 74 mg/kg, 76 mg/kg, 78 mg/kg, 80 mg/kg, 82 mg/kg, 84 mg/kg, 86 mg/kg, 88 mg/kg, 90 mg/kg, 92 mg/kg, 94 mg/kg, 96 mg/kg, 98 mg/kg, or up to about 100 mg/kg body weight of a subject. In aspects, cyclophosphamide can be administered from about 40 mg/kg to about 50 mg/kg of a subject. cyclophosphamide can be administered to a subject over at least about 2 days to about 5 days. In some cases, a cyclophosphamide can be administered from about 10 mg/kg to about 15 mg/kg of a subject. In some cases, cyclophosphamide can be administered to a subject over at least about 7 days to about 10 days. In some cases, cyclophosphamide can be administered from about 3 mg per kg to about 5 mg per kg of a subject. In some cases, cyclophosphamide can be administered from about 50 mg per kg to about 80 mg/kg of a subject. In some cases, cyclophosphamide can be administered in excess of 50 mg per kg. In some cases, cyclophosphamide can be administered at about 60 mg per kg. In aspects, cyclosporine can be administered from about 15 mg/kg of body weight per day. In aspects, azathioprine can be administered from about 1 mg/kg of body weight per day given as a single dose once a day or divided into two doses. In aspects, tacrolimus can be administered from about 0.1 to 0.2 mg/kg body weight.

In aspects, a conventional therapy can be administered from at least about or at most about: 1 mg, 2 mg, 3 mg, 4 mg, 6 mg, 8 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, 40 mg, 42 mg, 44 mg, 46 mg, 48 mg, 50 mg, 52 mg, 54 mg, 56 mg, 58 mg, 60 mg, 62 mg, 64 mg, 66 mg, 68 mg, 70 mg, 72 mg, 74 mg, 76 mg, 78 mg, 80 mg, 82 mg, 84 mg, 86 mg, 88 mg, 90 mg, 92 mg, 94 mg, 96 mg, 98 mg, or up to about 100 mg. In aspects, methotrexate can be administered from about 25 to 75 milligrams once a week. In aspects, methotrexate can be administered from about 2.5 milligrams 2 to 4 times a week. In aspects, prednisone is administered from about 10 to 60 mg/day given in a single daily dose or in 2 to 4 divided doses. In aspects, prednisone is administered at about 2.5 to 10 mg/day. In aspects, prednisone is administered at about 1 to 1.5 mg/kg/day.

In aspects, a therapeutic regimen comprises administering an anti-CD19 antibody or antigen-binding fragment thereof. In aspects, a therapeutic regimen comprises administering 300 mg of inebilizumab. In aspects, a therapeutic regimen comprises administering about 100-300 mg, 150-300 mg, 200-400 mg, 250-500 mg, 300-500 mg of inebilizumab.

Any of the dosages provided may be effective dosages for a method comprising prevention, reduction, or elimination. The dosages provided can also be effective at stabilizing, reducing, or eliminating disease or a symptom thereof as described herein.

### Dosing Frequency

In aspects, an anti-CD19 antibody or antigen-binding fragment thereof of the disclosure is administered on a schedule that provides optimal results. In aspects, an anti-CD19 antibody or antigen-binding fragment thereof disclosed herein is administered to a subject in need thereof about once a week, about twice a week, about every two weeks, about every four weeks, about once a month, about every six weeks, about every two months, about every 3 months, about every 12 weeks, about every fifteen weeks, about every sixteen weeks, about every four months, about every five months, about every six months, or semiannually.

Any number of administrations may be provided to a subject in need thereof. In aspects, a subject is administered an effective dose daily, weekly, biweekly, or up to about the lifetime of the subject. In aspects, administration are given for up to about 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 1 year, 2 years, 3 years, 4 years, or 5 years. In aspects, a subject receives an effective dose on day 1, day 15, day 183, day 365, day 547, day 729, and day 911 post treatment initiation. In aspects, a subject receives an effective dose on week 0, week 2, week 26, week 52, week 78, week 104, week 130, and week 156 post treatment initiation. In aspects, a subject receives an effective dose of 300 mg of an anti-CD19 antibody or antigen-binding fragment thereof on day 1, day 15, day 183, day 365, day 547, day 729, and day 911 post treatment initiation. In aspects, a subject receives an effective dose of 300 mg of an anti-CD19 antibody or antigen-binding fragment thereof on week 0, week 2, week 26, week 52, week 78, week 104, and week 130 post treatment initiation. In aspects, a subject receives an effective dose of 300 mg of an anti-CD19 antibody or antigen-binding fragment thereof on week 0, week 2, week 26, week 52, week 78, week 104, week 130, and week 156 post treatment initiation. In aspects, a subject in need thereof is administered an effective dose of 300 mg of an anti-CD19 antibody or antigen-binding fragment thereof on day 1, day 15, and then every 6 months (or 24 weeks) thereafter as needed. Any of the aforementioned administrations can deviate by about 0-7 days, 0-5 days, 0-4 days, 0-3 days, 0-2 days, by about 3 days, or by about 7 days.

In aspects, a subject receives an effective dose of an anti-CD19 antibody or antigen-binding fragment thereof on day 1, day 15±3 days, day 183±7 days, day 365±7 days, and day 547±7 days post treatment initiation. In aspects, a subject receives an effective dose of an anti-CD19 antibody or antigen-binding fragment thereof on day 1, day 15±3 days, day 183±7 days, day 365±7 days, day 547±7 days, and day 729±7 days post treatment initiation. In aspects, a subject receives an effective dose of an anti-CD19 antibody or antigen-binding fragment thereof on day 1, day 15±3 days, day 183±7 days, day 365±7 days, day 547±7 days, day 729±7 days, and day 911±7 days post treatment initiation. In aspects, a subject receives an effective dose of an anti-CD19 antibody or antigen-binding fragment thereof on week 0, week 2±3 days, week 26±3 days, week 52±7 days, week 78±7 days, week 104±7 days, and week 130±7 days post treatment initiation. In aspects, a subject receives an effective dose of an anti-CD19 antibody or antigen-binding fragment thereof on week 0, week 2±3 days, week 26±3 days, week 52±7 days, week 78±7, week 104±7 days, week 130±7 days, and week 156±7 days post treatment initiation.

In aspects, an anti-CD19 antibody or antigen-binding fragment thereof is administered at an effective single dose. In aspects, a subject in need thereof is administered at least two administrations of an anti-CD19 antibody or antigen-binding fragment thereof.

In aspects, an anti-CD19 antibody or antigen-binding fragment thereof is administered at a dose of about 300 mg for at least 1 dose and is administered about once every 6 months thereafter. In aspects, an anti-CD19 antibody or antigen-binding fragment thereof is administered at a dose of about 300 mg for at least 2 doses and is administered about once every 6 months thereafter. In aspects, an anti-CD19 antibody or antigen-binding fragment thereof is administered at a dose of about 300 mg once about every two weeks, once about every month, once about every two months, once about every three months, once every six months, or one every 12 months. In aspects, an anti-CD19 antibody or antigen-binding fragment thereof is administered at a dose of about 300 mg once every two weeks for 2 doses and then once every 6 months thereafter for at least 1, 2, 3, 4, 5, 6, 7, 8, 9, 10, 11, 12, 13, 14 or up to at least 15 doses thereafter.

In aspects, an anti-CD19 antibody or antigen-binding fragment thereof is administered for two or more doses. In aspects, an anti-CD19 antibody or antigen-binding fragment thereof of the disclosure is administered to a subject in need thereof until myositis is prevented, reduced, or eliminated as determined by a test of the disclosure. In aspects, an anti-CD19 antibody or antigen-binding fragment thereof is administered as an effective dose of about 300 mg. In aspects, a method disclosed herein comprises administration of an antibody or antigen-binding fragment thereof that binds CD19 to a subject in need thereof, wherein the antibody or antigen-binding fragment thereof is administered at an effective dose of about 300 mg intravenously. In aspects, a method disclosed herein comprises administration of an antibody or antigen-binding fragment thereof that binds CD19 to a subject in need thereof, wherein the antibody or antigen-binding fragment thereof is administered as a single dose of about 300 mg every 2 weeks for at least 2 doses and is then administered about once every 6 months thereafter. In aspects, a method disclosed herein comprises administration of an antibody or antigen-binding fragment thereof that binds CD19 to a subject in need thereof, wherein the antibody or antigen-binding fragment thereof is administered as a single dose of about 300 mg every 2 weeks for at least 2 doses and is then administered about once every 6 months thereafter. In aspects, a method disclosed herein comprises administration of an antibody or antigen-binding fragment thereof that binds CD19 to a subject in need thereof, wherein the antibody or antigen-binding fragment thereof is administered as a single dose of about 300 mg every 2 weeks for at least 2 doses and is then administered about once every 6 months thereafter for 2, 3, 4, 5, 6, 7, 8, 9, or 10, or more, doses. In aspects, a method disclosed herein comprises administration of an antibody or antigen-binding fragment thereof that binds CD19 to a subject in need thereof, wherein the antibody or antigen-binding fragment thereof is administered as a single dose of about 300 mg every 2 weeks for at least 2 administrations and is administered then about once every 6 months thereafter intravenously. In aspects, a method disclosed herein comprises administration of an antibody or antigen-binding fragment thereof that binds CD19 to a subject in need thereof, wherein the antibody or antigen-binding fragment thereof is administered as a single dose of about 300 mg every 2 weeks for at least 2 administrations and is administered then about once every 6 months thereafter for 2, 3, 4, 5, 6, 7, 8, 9, or 10, or more, doses intravenously.

In aspects, a method disclosed herein comprises administration of an antibody or antigen-binding fragment thereof that binds CD19 to a subject in need thereof, wherein the antibody or antigen-binding fragment thereof comprise HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of: SEQ ID NOs: 2, 3, 4, 6, 7, and 8, respectively. In aspects, a method disclosed herein comprises administration of an antibody or antigen-binding fragment thereof that binds CD19 to a subject in need thereof, wherein the antibody or antigen-binding fragment thereof comprise HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of: SEQ ID NOs: 2, 3, 4, 6, 7, and 8, respectively, and wherein the antibody or antigen-binding fragment thereof is administered at a dose of about 300 mg for at least 2 doses and is then administered about once every 6 months thereafter. In aspects, a method disclosed herein comprises administration of an antibody or antigen-binding fragment thereof that binds CD19 to a subject in need thereof, wherein the antibody or antigen-binding fragment thereof comprise HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of: SEQ ID NOs: 2, 3, 4, 6, 7, and 8, respectively, and wherein the antibody or antigen-binding fragment thereof is administered at a dose of about 300 mg for at least 2 doses and is then administered about once every 6 months thereafter for 2, 3, 4, 5, 6, 7, 8, 9, or 10, or more, doses. In aspects, a method disclosed herein comprises administration of an antibody or antigen-binding fragment thereof that binds CD19 to a subject in need thereof, wherein the antibody or antigen-binding fragment thereof comprise HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of: SEQ ID NOs: 2, 3, 4, 6, 7, and 8, respectively, and wherein the antibody or antigen-binding fragment thereof is administered at a dose of about 300 mg once every two weeks for 2 doses and is then administered about once every 6 months thereafter intravenously. In aspects, a method disclosed herein comprises administration of an antibody or antigen-binding fragment thereof that binds CD19 to a subject in need thereof, wherein the antibody or antigen-binding fragment thereof comprise HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of: SEQ ID NOs: 2, 3, 4, 6, 7, and 8, respectively, and wherein the antibody or antigen-binding fragment thereof is administered at a dose of about 300 mg once every two weeks for 2 doses and is then administered about once every 6 months thereafter for 2, 3, 4, 5, 6, 7, 8, 9, or 10, or more, doses intravenously.

In aspects, a method disclosed herein comprises administration of an antibody or antigen-binding fragment thereof that binds CD19 to a subject in need thereof, wherein the antibody or antigen-binding fragment thereof comprise VH and VL, wherein the VH and VL comprise the amino acid sequences having at least about 80%, 85%, 90%, 95%, 99%, or 100% identity SEQ ID NOs: 1 and 5, respectively, and wherein the antibody or antigen-binding fragment is administered as a dose of about 300 mg. In aspects, a method disclosed herein comprises administration of an antibody or antigen-binding fragment thereof that binds CD19 to a subject in need thereof, wherein the antibody or antigen-binding fragment thereof comprise VH and VL, wherein the VH and VL comprise the amino acid sequences of: SEQ ID NOs: 1 and 5, respectively, and wherein the antibody or antigen-binding fragment thereof is administered as a dose of about 300 mg intravenously. In aspects, a method disclosed herein comprises administration of an antibody or antigen-binding fragment thereof that binds CD19 to a subject in need thereof, wherein the antibody or antigen-binding fragment thereof comprise VH and VL, wherein the VH and VL comprise the amino acid sequences of: SEQ ID NOs: 1 and 5, respectively, and wherein the antibody or antigen-binding fragment thereof is administered as a dose of about 300 mg once every 2 weeks for 2 doses and is then administered about every 6 months thereafter. In aspects, a method disclosed herein comprises administration of an antibody or antigen-binding fragment thereof that binds CD19 to a subject in need thereof, wherein the antibody or antigen-binding fragment thereof comprise VH and VL, wherein the VH and VL comprise the amino acid sequences of: SEQ ID NOs: 1 and 5, respectively, and wherein the antibody or antigen-binding fragment thereof is administered as a dose of about 300 mg once every 2 weeks for 2 doses and is then administered about every 6 months thereafter for 2, 3, 4, 5, 6, 7, 8, 9, or 10, or more, doses. In aspects, a method disclosed herein comprises administration of an antibody or antigen-binding fragment thereof that binds CD19 to a subject in need thereof, wherein the antibody or antigen-binding fragment thereof comprise VH and VL, wherein the VH and VL comprise the amino acid sequences of: SEQ ID NOs: 1 and 5, respectively, and wherein the antibody or antigen-binding fragment thereof is administered as a dose of about 300 mg once every 2 weeks for 2 doses and is then administered about once every 6 months for doses thereafter intravenously. In aspects, a method disclosed herein comprises administration of an antibody or antigen-binding fragment thereof that binds CD19 to a subject in need thereof, wherein the antibody or antigen-binding fragment thereof comprise VH and VL, wherein the VH and VL comprise the amino acid sequences of: SEQ ID NOs: 1 and 5, respectively, and wherein the antibody or antigen-binding fragment thereof is administered as a dose of about 300 mg once every 2 weeks for 2 doses and is then administered about once every 6 months thereafter for 2, 3, 4, 5, 6, 7, 8, 9, or 10, or more, doses intravenously.

In aspects, a subject in need thereof is administered an effective dose of an anti-CD19 antibody or antigen-binding fragment thereof once about every 2-4 weeks. In aspects, a subject in need thereof is administered an effective dose of an anti-CD19 antibody or antigen-binding fragment thereof once about every 24 weeks or 6 months. In aspects, a subject in need thereof is administered an effective dose of an anti-CD19 antibody or antigen-binding fragment thereof once every 2 weeks for 2 doses and then every 6 months thereafter. In aspects, a subject in need thereof is administered an effective dose of an anti-CD19 antibody or antigen-binding fragment thereof once every 2 weeks for 2 doses and then every 6 months thereafter for 2, 3, 4, 5, 6, 7, 8, 9, or 10, or more, doses. In aspects, a subject in need thereof is administered 300 mg of an anti-CD19 antibody or antigen-binding fragment thereof once every 2 weeks for at least 2 doses, once every 6 months for at least 1 dose, once every 6 months for at least 2 doses, once every 6 months for at least 3 doses, once every 6 months for at least 4 doses, or once every 6 months thereafter for 2, 3, 4, 5, 6, 7, 8, 9, or 10, or more, doses. In aspects, an anti-CD19 antibody or antigen-binding fragment thereof is administered at an effective dose of about 300 mg once every two weeks. In aspects, an anti-CD19 antibody or antigen-binding fragment thereof is administered at an effective dose of about 300 mg every 6 months.

### Methods

In aspects herein, methods are directed to treat, reduce, or eliminate a myositis disease or disorder. In aspects, a method comprises administering an anti-CD19 antibody or antigen-binding fragment thereof of the disclosure. In aspects, an anti-CD19 antibody or antigen-binding fragment thereof is administered to prevent, reduce, or eliminate myositis in a subject in need thereof using any of the dosing schedules disclosed herein. In aspects, a myopathy disease or disorder comprises immune-mediated necrotizing myopathy (IMNM). In aspects, an anti-CD19 antibody or antigen-binding fragment thereof is administered to prevent, reduce, or eliminate IMNM in a subject in need thereof using any of the dosing schedules disclosed herein. In aspects, an anti-CD19 antibody or antigen-binding fragment thereof is administered to prevent, reduce, or eliminate disease in a subject that tests positive for anti-signal recognition particle (e.g., anti-SRP) antibodies prior to treatment. In aspects, an anti-CD19 antibody or antigen-binding fragment thereof is administered to prevent, reduce, or eliminate disease in a subject that tests positive for anti-3-hydroxy-3-methylglutaryl-coenzyme A reductase (e.g., anti-HMGCR) antibodies prior to treatment. In aspects, an anti-CD19 antibody or antigen-binding fragment thereof is administered to prevent, reduce, or eliminate disease in a subject that has an IMNM diagnosis and tests positive for anti-signal recognition particle (e.g., anti-SRP) antibodies prior to treatment. In aspects, an anti-CD19 antibody or antigen-binding fragment thereof is administered to prevent, reduce, or eliminate disease in a subject that has an IMNM diagnosis and tests positive for anti-3-hydroxy-3-methylglutaryl-coenzyme A reductase (e.g., anti-HMGCR) antibodies prior to treatment.

In aspects, a method of treating a subject in need thereof comprises administering an effective amount of an anti-CD19 antibody or antigen-binding fragment thereof. In aspects, an administration is effective in reducing a disease or a disorder as compared to: a) the disease or disorder in an otherwise comparable subject lacking the administration; or b) a baseline measurement of the disease or disorder in the subject in need thereof. In aspects, an administration is effective in reducing a level of CD19+ B cells in a subject in need thereof as compared to a baseline level of the subject. In aspects, a level of CD19+ B cells is reduced in a subject administered an anti-CD19 antibody or antigen-binding fragment by at least about or at most about: 3-fold, 5-fold, 10-fold, 25-fold, 50-fold, 75-fold, 100-fold, 150-fold, or 250-fold as compared to a baseline level of the subject. In aspects, a level of CD19+ B cells is reduced in a subject administered an anti-CD19 antibody or antigen-binding fragment by at least about or at most about: 5%, 10%, 15%, 20%, 25%, 30%, 40%, 50%, 60%, 70%, 80%, 90% or up to about 100% as compared to a baseline level of the subject.

In aspects, an anti-CD19 antibody or antigen-binding fragment thereof is used to treat a myositis disease or disorder. In aspects, an anti-CD19 antibody or antigen-binding fragment thereof is used to treat a subject in need thereof using any of the dosing schedules disclosed herein.

In aspects, an anti-CD19 antibody or antigen-binding fragment thereof is administered at a dose of about 300 mg. In aspects, an anti-CD19 antibody or antigen-binding fragment thereof is administered at a dose of about 300 mg intravenously. In aspects, an anti-CD19 antibody or antigen-binding fragment thereof is administered at a dose of about 300 mg every 2 weeks. In aspects, an anti-CD19 antibody or antigen-binding fragment thereof is administered at a dose of about 300 mg every 6 months. In aspects, an anti-CD19 antibody or antigen-binding fragment thereof is administered at a dose of about 300 mg every 2 weeks for at least 2 doses. In aspects, an anti-CD19 antibody or antigen-binding fragment thereof is administered at a dose of about 300 mg every 6 months for at least or at most 4 doses. In aspects, an anti-CD19 antibody or antigen-binding fragment thereof is administered at a dose of about 300 mg every 2 weeks for at least 2 doses and then every 6 months thereafter for at least or at most 4 doses. In aspects, an anti-CD19 antibody or antigen-binding fragment thereof is administered at a dose of about 300 mg via intravenous administration every 2 weeks for at least 2 doses. In aspects, an anti-CD19 antibody or antigen-binding fragment thereof is administered at a dose of about 300 mg via intravenous administration every 6 months for at least or at most 4 doses. In aspects, an anti-CD19 antibody or antigen-binding fragment thereof is administered at a dose of about 300 mg via intravenous administration every 2 weeks for at least 2 doses and then every 6 months thereafter for at least or at most 4 doses. In aspects, an anti-CD19 antibody or antigen-binding fragment thereof is administered at a dose of about 300 mg via intravenous administration every 2 weeks for at least 2 doses and then every six months thereafter.

In aspects, a composition comprising an anti-CD19 antibody or antigen-binding fragment is formulated for administration. In aspects, an anti-CD19 antibody or antigen-binding fragment thereof is formulated at a concentration of at least about or at most about: 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, 200 mg/mL, 205 mg/mL, 210 mg/mL, 215 mg/mL, 220 mg/mL, 225 mg/mL, 230 mg/mL, 235 mg/mL, 240 mg/mL, 245 mg/mL, 250 mg/mL, 255 mg/mL, 260 mg/mL, 265 mg/mL, 270 mg/mL, 275 mg/mL, 280 mg/mL, 285 mg/mL, 290 mg/mL, 295 mg/mL, or up to about 300 mg/mL. In aspects, an anti-CD19 antibody or antigen-binding fragment thereof effective dose may be formulated at a concentration of about 10 mg/mL.

The dose and dosing regimen of an anti-CD19 antibody or antigen-binding fragment thereof disclosed herein may be such that any therapeutic effect achieved from administration of an anti-CD19 antibody or antigen-binding fragment thereof to treat any myopathy disease or disorder, may be considered to be “long-lasting.” A “long-lasting” effect of an anti-CD19 antibody or antigen-binding fragment thereof in the treatment of a myositis disease or disorder is one in which the therapeutic effect achieved by an anti-CD19 antibody or antigen-binding fragment thereof is maintained (although an anti-CD19 antibody or antigen-binding fragment thereof is no longer administered) over at least 4 weeks, at least 6 weeks, at least 8 weeks, at least 10 weeks, at least 12 weeks, at least 16 weeks, at least 20 weeks, at least 24, at least 28 weeks, at least 32 weeks, at least 36 weeks, at least 40 weeks, at least 44 weeks, at least 48 weeks, at least 1 year, at least 2 years, at least 3 years, at least 4 years or up to about at least 5 years following a prior administration of an anti-CD19 antibody or antigen-binding fragment thereof.

In aspects, administration of an anti-CD19 antibody or antigen-binding fragment thereof is effective in eliminating disease in a subject in need thereof. In aspects, administration of an anti-CD19 antibody or antigen-binding fragment thereof is effective in reducing disease in a subject in need thereof. In some cases, administration of an anti-CD19 antibody or antigen-binding fragment thereof is effective in eliminating or reducing disease in a subject in need thereof for at least about 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, at least about 5 years, or for the lifetime of the subject. In aspects, administration of an anti-CD19 antibody or antigen-binding fragment thereof is effective in eliminating disease in a subject in need thereof for at least about 1 week, 2 weeks, 3 weeks, 4 weeks, 5 weeks, 6 weeks, 7 weeks, 8 weeks, 9 weeks, 10 weeks, 11 weeks, or at least about 12 weeks. In aspects, administration of an anti-CD19 antibody or antigen-binding fragment thereof is effective in eliminating or reducing disease in a subject in need thereof for at least about 1 month, 2 months, 3 months, 4 months, 5 months, 6 months, 7 months, 8 months, 9 months, 10 months, 11 months, or at least about 12 months. In aspects, administration of an anti-CD19 antibody or antigen-binding fragment thereof is effective in eliminating or reducing disease in a subject in need thereof for at least about 1 year, 2 years, 3 years, 4 years, 5 years, 6 years, 7 years, 8 years, 9 years, or at least about 10 years. In aspects, administration of an anti-CD19 antibody or antigen-binding fragment thereof is effective in eliminating or reducing disease for the lifetime of a subject in need thereof.

In aspects, a subject in need thereof has been administered one or more conventional therapies for the treatment of a myositis disease or disorder prior to the administration of an anti-CD19 antibody or antigen-binding fragment thereof. In aspects, a conventional therapy comprises a corticosteroid, an immunomodulatory agent, and/or an anti-pain agent. In aspects, a subject continues on a conventional therapy while being administered an anti-CD19 antibody or antigen-binding fragment thereof. In aspects, a conventional therapy is administered as a second therapy. In aspects, any of the referenced therapies may be combined in a therapeutic regimen for the treatment of a myositis disease or disorder. In aspects, a subject is treated with both an anti-CD19 antibody or antigen-binding fragment thereof and a conventional therapy.

Methods of the disclosure can comprise tapering of one or more conventional therapies and/or an anti-CD19 antibody or antigen-binding fragment thereof. In aspects, a method comprises tapering of a conventional therapy. In aspects, a method comprises a tapering of a corticosteroid. In aspects, a method comprises a tapering of methylprednisolone. In aspects, a method comprises a tapering of prednisone. The tapering can occur at any time. In aspects, a tapering occurs before administration of an anti-CD19 antibody or antigen-binding fragment thereof. In aspects, a tapering occurs concurrent with administration of an anti-CD19 antibody or antigen-binding fragment thereof. In aspects, a tapering occurs after administration of an anti-CD19 antibody or antigen-binding fragment thereof. In aspects, a tapering comprises a reduction in frequency of use at least about 10%, 15%, 20%, 25%, 30%, 40%, or 50%. In aspects, a tapering is a decrease from about a ≥10 mg/day dosage to a 7.5 mg/day dosage. In aspects, a tapering occurs about every 2 weeks beginning at week 26.

### Corticosteroid

In aspects, a subject is administered a corticosteroid. In aspects, a subject is administered a corticosteroid in combination with an anti-CD19 antibody or antigen-binding fragment thereof as disclosed in any of the methods herein. In aspects, a corticosteroid comprises a glucocorticoid. Glucocorticoids (GCs) are steroid hormones used for the treatment of inflammation, a myositis disease or disorder, and/or cancer. To exert their broad physiological and therapeutic effects, GCs bind to the GC receptor (GR) which belongs to the nuclear receptor superfamily of transcription factors. In aspects, one or more GCs are administered to a subject in need thereof. In aspects, one or more GCs is selected from the group consisting of, but not limited to, triamcinolone, methylprednisolone, budesonide, dexamethasone, triamcinolone, prednisone, hydrocortisone, dexamethasone, betamethasone, prednisolone, deflazacort, and combinations thereof. In aspects, a GC is prednisone. In aspects, a GC is methylprednisolone.

### Immunomodulatory Agent

In aspects, a subject is administered an immunomodulatory agent. In aspects, an immunomodulatory agent is a non-steroidal immunosuppressant (IST). In aspects, a subject is administered an immunomodulatory agent in combination with an anti-CD19 antibody or antigen-binding fragment thereof as disclosed in any of the methods herein. Immunomodulatory agents comprise methotrexate, cyclophosphamide, azathioprine, mycophenolate mofetil, mycophenolic acid, tacrolimus, cyclosporine, sirolimus, everolimus, and combinations thereof. In aspects, an immunomodulatory agent is methotrexate. In aspects, an immunomodulatory agent is cyclophosphamide. In aspects, an immunomodulatory agent is azathioprine. In aspects, an immunomodulatory agent is mycophenolate mofetil. In aspects, an immunomodulatory agent is mycophenolic acid. In aspects, an immunomodulatory agent is tacrolimus. In aspects, an immunomodulatory agent is cyclosporine.

### Anti-Pain Agent

In aspects, a subject is administered an anti-pain agent. In aspects, a subject is administered an anti-pain agent in combination with an anti-CD19 antibody or antigen-binding fragment thereof as disclosed in any of the methods herein. Anti-pain agents comprise NSAIDs, acetaminophen, corticosteroid, opioid (e.g., codeine, fentanyl, Vicodin, morphine, oxycodone, Percocet), anti-depressant, anti-histamine (e.g., diphenhydramine or cetirizine, and the like) anti-convulsant, lidocaine, and combinations thereof. In aspects, an anti-pain agent is acetaminophen. In aspects, an anti-pain agent is an anti-histamine.

In aspects, any of the conventional therapies are combined as part of a treatment regimen or combination therapy. In aspects, an anti-CD19 antibody or antigen-binding fragment thereof is administered sequentially with any therapy disclosed herein. In aspects, an anti-CD19 antibody or antigen-binding fragment thereof may generally be administered concurrently with any second therapy disclosed herein. In aspects, a subject in need thereof has been administered any conventional therapy for the treatment of a myositis disease or disorder prior to the administration of an anti-CD19 antibody or antigen-binding fragment thereof.

In aspects, one or more conventional therapies are administered for about 1, about 2, about 3, about 4, about 5, about 6, about 7, about 8, about 9, about 10, about 11, about 12, about 13, about 14, about 15, about 20, about 25, about 30, about 35, about 40, about 52, about 52 weeks or more prior to the administration of an anti-CD19 antibody or antigen-binding fragment thereof. In aspects, one or more conventional therapies are administered for about 2 weeks prior to the administration of an anti-CD19 antibody or antigen-binding fragment thereof. In aspects, one or more conventional therapies are administered for about 12 weeks prior to the administration of an anti-CD19 antibody or antigen-binding fragment thereof.

A dose and dosing regimen of an anti-CD19 antibody or antigen-binding fragment thereof as disclosed herein may be such that any therapeutic effect achieved from administration of an anti-CD19 antibody or antigen-binding fragment thereof to treat any myopathy disease or disorder, may be considered to be “long-lasting.” A “long-lasting” effect of an anti-CD19 antibody or antigen-binding fragment thereof in a treatment of a myopathy disease or disorder is one in which a therapeutic effect achieved by an anti-CD19 antibody or antigen-binding fragment thereof is maintained (although the anti-CD19 antibody or antigen-binding fragment thereof is no longer administered) over at least 4 weeks, at least 6 weeks, at least 8 weeks, at least 10 weeks, at least 12 weeks, at least 16 weeks, at least 20 weeks, least 24 weeks, at least 28 weeks, at least 32 weeks, at least 36 weeks, at least 40 weeks, at least 44 weeks, at least 48 weeks, at least 52, at least 56 weeks, at least 60 weeks, at least 64 weeks, at least 68 weeks, at least 72 weeks, at least 76 weeks, at least 80 weeks, at least 84 weeks, at least 88 weeks, at least 92 weeks, at least 96 weeks, at least 100 weeks, or at least 104 weeks following administration of the last dose of a course of an anti-CD19 antibody or antigen-binding fragment thereof. In aspects, less frequent dosing of any of the compositions provided herein may be advantageous. Exemplary advantages of less frequent dosing include but are not limited to reduced frequency of side effects associated with an administered composition, reduced treatment-associated toxicity, increased quality of life for treated subjects, and the like.

### Assessment

In aspects, a subject is assessed. In aspects, a subject is assessed as part of a treatment. In aspects, a subject having a confirmed or probable myopathy disease or disorder is assessed as part of a treatment. An assessment can occur at any point before, during, or after administration with an anti-CD19 antibody or antigen-binding fragment thereof. In aspects, an assessment is performed before an administration initiates. In aspects, an assessment is performed concurrent with an administration. In aspects, an assessment is performed after an administration finishes.

In aspects, an assessment can occur at any point before, during or after administration with an anti-CD19 antibody or antigen-binding fragment thereof as part of a Phase 2 trial. In aspects, an assessment can occur at any point before, during or after administration with an anti-CD19 antibody or antigen-binding fragment thereof as part of a Phase 3 trial. In aspects, an assessment can occur at any point before, during or after administration with an anti-CD19 antibody or antigen-binding fragment thereof as part of an Open Label Period (OLP, also known as Open Label Extension; OLE).

In aspects, a subject is assessed, and a baseline measurement of disease is determined. In aspects, a subject is assessed, and a baseline measurement of disease is determined at any point before, during, or after administration with an anti-CD19 antibody or antigen-binding fragment thereof. In aspects, an assessment is performed, and a baseline measurement of disease is determined before an administration with an anti-CD19 antibody or antigen-binding fragment thereof. In aspects, an assessment is performed, and a baseline measurement of disease is determined concurrent with an administration of an anti-CD19 antibody or antigen-binding fragment thereof. In aspects, an assessment is performed, and a baseline measurement of disease is determined after an administration of an anti-CD19 antibody or antigen-binding fragment thereof.

In aspects, a baseline measurement of disease increases or decreases in response to an administration of the disclosure or a lack thereof. In aspects, a baseline measurement of disease may increase and indicate effective treatment of a disease or disorder. In aspects, a baseline measurement of disease may decrease and indicate effective treatment of a disease or disorder. In aspects, a baseline measurement of disease increases or decreases after administration with an anti-CD19 antibody or antigen-binding fragment thereof. In aspects, a baseline measurement of disease may increase after administration with an anti-CD19 antibody or antigen-binding fragment and indicate effective treatment of a disease or disorder. In aspects, a baseline measurement of disease may decrease after administration with an anti-CD19 antibody or antigen-binding fragment and indicate effective treatment of a disease or disorder.

In aspects, a baseline measurement of disease is compared to the measurement of disease of the subject after administration of the anti-CD19 antibody or antigen-binding fragment thereof. In aspects, a measurement of disease of a subject administered an anti-CD19 antibody or antigen-binding fragment thereof increases as compared to a baseline measurement of disease measured prior to administration with an anti-CD19 antibody or antigen-binding fragment. In aspects, a measurement of disease of a subject administered an anti-CD19 antibody or antigen-binding fragment thereof decreases as compared to a baseline measurement of disease measured prior to administration with an anti-CD19 antibody or antigen-binding fragment. In aspects, a measurement of disease of a subject administered an anti-CD19 antibody or antigen-binding fragment thereof increases as compared to a baseline measurement of disease measured prior to administration with an anti-CD19 antibody or antigen-binding fragment and indicates effective treatment. In aspects, a measurement of disease of a subject administered an anti-CD19 antibody or antigen-binding fragment thereof decreases as compared to a baseline measurement of disease measured prior to administration with an anti-CD19 antibody or antigen-binding fragment and indicates effective treatment.

In aspects an assessment comprises determining treatment efficacy. Efficacy can be determined by any of the assessments of the disclosure. In aspects, effectiveness at preventing, reducing, or eliminating a myopathy disease or disorder (e.g., IMNM) is determined by detecting a change in baseline in a subject's blood level of B cells. In aspects, effectiveness at preventing, reducing, or eliminating a myopathy disease or disorder is determined evaluating: pharmacokinetic parameters of an anti-CD19 antibody or antigen-binding fragment thereof used to a treat a subject in need thereof, a change in baseline in a subject's blood level of B cells, a presence of anti-drug antibodies (ADA), and combinations thereof. In aspects, effectiveness at preventing, reducing, or eliminating a myopathy disease or disorder is determined by determining pharmacokinetic parameters of an anti-CD19 antibody or antigen-binding fragment thereof used to a treat a subject in need thereof. In aspects, effectiveness at preventing, reducing, or eliminating a myopathy disease or disorder is determined by determining a change in baseline in a subject's blood level of B cell. In aspects, effectiveness at preventing, reducing, or eliminating a myositis disease or disorder is determined by determining a presence of ADA. In aspects, a myositis disease or disorder comprises IMNM.

Any of the below-referenced assessments can occur at any time. In aspects, a subject is assessed by the minute, hourly, daily, weekly, monthly, quarterly, or yearly. In aspects, an assessment is completed twice daily, biweekly, bimonthly, or semiannually.

In aspects, an assessment is performed from day−28, −27, −26, −25, −24, −23, −22, −21, −20, −19, −18, −17, −16, −15, −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, or up to about day 813 post treatment. Timing of assessments are further described below.

### Assessment of Inclusion

In aspects, a subject in need thereof is assessed prior to beginning treatment. An exemplary assessment or inclusion criterion comprises a subject having one or more myopathy diseases or disorders.

In aspects, a subject in need thereof may be either male or female. In aspects, a subject in need thereof is an adult subject. In aspects, a subject in need thereof may be aged between about 16 and 80 years of age.

In aspects, a subject in need thereof may have a myopathy diagnosis that comprises, but is not limited to, immune-mediated necrotizing myopathy (IMNM). In aspects, a subject in need thereof has a diagnosis of IMNM. In aspects, a subject in need thereof has a diagnosis of IMNM and has tested positive for anti-single particle receptor (anti-SPR) or anti-3-hydroxy-3-methyl-glutaryl-coenzyme A reductase (anti-HMGCR) antibodies prior to treatment initiation. In aspects, a subject in need thereof has a creatine kinase (CK) level >1000 U/L. In aspects, a subject in need thereof has a manual muscle testing-8 (MMT-8) score of ≤136 units.

In aspects, a subject in need thereof has a diagnosis of definite or probable myopathy according to American College of Rheumatology (ACR) criteria. In aspects, a subject in need thereof has a diagnosis of definite or probable myopathy according to European League Against Rheumatism (EULAR) criteria. In aspects, a subject in need thereof has a diagnosis of definite or probable myositis according to 2016 ACR/EULAR total improvement score (TIS) criteria.

In aspects, a subject in need thereof may have prior and/or concomitant therapy for treatment of a myopathy disorder at the time of treatment initiation. In aspects, a subject in need thereof may have stable dosing of methotrexate, azathioprine, mycophenolate mofetil, mycophenolic acid, tacrolimus, and/or cyclosporine for at least 12 weeks prior to treatment initiation. In aspects, a subject in need thereof may have stable dosing of corticosteroids (e.g., prednisone or methylprednisolone) for at least 2 weeks prior to treatment initiation.

### 2016 American College of Rheumatology/European League Against Rheumatism (ACR/EULAR) Total Improvement Score (TIS)

In aspects, an assessment comprises a 2016 ACR/EULAR TIS. In aspects, a 2016 ACR/EULAR TIS is a validated method for assessing myopathies. In aspects, 2016 ACR/EULAR TIS may be scored from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and up to about 100. In aspects, 2016 ACR/EULAR TIS is from about 1-10, about 10-30, about 20-40, about 30-50, about 40-60, about 50-70, about 60-80, about 70-90, or about 80-100. The 2016 ACR/EULAR TIS may be ≥20, ≥30, ≥40, ≥50, or >60. In aspects, the criteria may be used as a continuous outcome measure using the 2016 ACR/EULAR TIS. In aspects, the criteria may be used as a categorical outcome of improvement (minimal, moderate, or major improvement). In aspects, the criteria may be used as both a continuous outcome measure and a categorical outcome of improvement, and thus are a hybrid measure. In aspects, a change from baseline is determined. In aspects, a subject's score is increased as compared to an otherwise comparable method wherein the subject of the otherwise comparable method is not administered an anti-CD19 antibody or antigen-binding fragment thereof.

In aspects, a 2016 ACR/EULAR TIS assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a 2016 ACR/EULAR TIS assessment to an anti-CD19 antibody or antigen-binding fragment thereof can be assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Week 2±3 days, Week 6±7 days, Week 12±7 days, Week 18±7 days, Week 22±7 days, Week 26±3 days, Week 30±3 days, Week 34±7 days, Week 38±7 days, Week 42±7 days, Week 46±7 days, Week 52±7 days, Week 78 weeks±7 days, and Week 104±7 days post treatment initiation.

### Manual Muscle Testing 8 (MMT8) Bilateral

In aspects, an assessment comprises MMT8 bilateral. In aspects, MMT8 bilateral is a set of 8 designated muscles tested bilaterally (MMT8, 2007). In aspects, axial (neck flexors) testing is included, so the potential MMT8 score is up to about at least 150. In aspects, MMT8 bilateral may be scored from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 148, or about 150. In aspects, MMT8 bilateral is from about 0-10, about 10-30, about 20-40, about 30-50, about 40-60, about 50-70, about 60-80, about 70-90, about 80-100, about 90-110, about 100-120, about 110-130, about 120-140, or about 130-150. In aspects, a MMT8 bilateral assessment may comprise assessing a subject in need thereof administered an anti-CD19 antibody or antigen-binding fragment thereof as compared to baseline. In aspects, a subject's MMT8 bilateral score is increased as compared to an otherwise comparable method wherein the subject of the otherwise comparable method is not administered an anti-CD19 antibody or antigen-binding fragment thereof. In aspects, administration of an anti-CD19 antibody or antigen-binding fragment thereof of the disclosure is effective in improving a MMT8 score in a subject by at least about or at most about 5%, 6%, 7%, 8%, 9%, 10%, 20%, 30%, 40%, 50%, 55%, 60%, 65%, 70%, 75%, 80%, 85%, 90%, 95%, 96%, 97%, 98%, 99%, or up to about 100% of the level prior the administration.

In aspects, a MMT8 bilateral assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a MMT8 bilateral assessment to an anti-CD19 antibody or antigen-binding fragment thereof can be assessed on about Day −28, Day −27, Day −26, Day −25, Day −24, Day −23, Day −22, Day −21, Day −20, Day −19, Day −18, Day −17, Day −16, Day −15, Day −14, Day −13, Day −12, Day −11, Day −10, Day −9, Day −8, Day −7, Day −6, Day −5, Day −4, Day −3, Day −2, Day −1, Day 0, Day 1, Week 2±3 days, Week 6±7 days, Week 12±7 days, Week 18±7 days, Week 22±7 days, Week 26±3 days, Week 30±3 days, Week 34±7 days, Week 38±7 days, Week 42±7 days, Week 46±7 days, Week 52±7 days, Week 78 weeks±7 days, and Week 104±7 days post treatment initiation.

### Pulmonary Function Tests (PFT)

In aspects, an assessment comprises a PFT. A PFT may assess lung function. Lung function may be assessed by measuring tidal volume (TV), minute volume (MV), vital capacity (VC), functional residual capacity (FRC), residual volume, total lung capacity, forced vital capacity (FVC), forced expiratory volume (FEV), forced expiratory flow (FEF), or peak expiratory flow rate (PEFR), or combinations thereof. In aspects, these assessments may be tested using spirometry or plethysmography, or a combination of both. In aspects, a PFT score has improved post treatment with a composition provided herein. In aspects, a PFT may comprise assessing a subject in need thereof administered an anti-CD19 antibody or antigen-binding fragment thereof as compared to baseline. In aspects, a subject's symptoms are improved as compared to an otherwise comparable method wherein the subject of the otherwise comparable method is not administered an anti-CD19 antibody or antigen-binding fragment thereof.

In aspects, a PFT to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PFT to an anti-CD19 antibody or antigen-binding fragment thereof can be assessed on about day −28, day −27, day −26, day −25, day −24, day −23, day −22, day −21, day −20, day −19, day −18, day −17, day −16, day −15, day −14, day −13, day −12, day −11, day −10, day −9, day −8, day −7, day −6, day −5, day −4, day −3, day −2, day −1, day 0, day 1, week 2±3 days, week 6±7 days, week 12±7 days, week 18±7 days, week 22±7 days, week 26±3 days, week 30±3 days, week 34±7 days, week 38±7 days, week 42±7 days, week 46±7 days, week 52±7 days, week 78 weeks±7 days, and week 104±7 days post treatment initiation.

### Timed Up and go (TUG) Test

In aspects, an assessment comprises a TUG test. A TUG test may assess mobility. In aspects, mobility may be assessed by observing a subject's stability, gait, stride length, or sway, or combinations thereof. In aspects, an assessment may be tested using a timed task. In aspects, a TUG tests a subject's ability to complete a timed task. In aspects, a subject who scores ≥12 seconds on a time task has scored poorly on the timed task. In aspects, a TUG test time has decreased, and thus improved, post treatment with a composition provided herein. In aspects, a TUG test may comprise assessing a subject in need thereof administered an anti-CD19 antibody or antigen-binding fragment thereof as compared to baseline. In aspects, a subject's score has improved as compared to an otherwise comparable method wherein the subject of the otherwise comparable method is not administered an anti-CD19 antibody or antigen-binding fragment thereof.

In aspects, a TUG assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a TUG assessment to an anti-CD19 antibody or antigen-binding fragment thereof can be assessed on about day 1, week 12±7 days, week 26±3 days, week 34±7 days, week 38±7 days, and week 46±7 days post treatment initiation.

### 2-Minute Walk Test (2MWT)

In aspects, an assessment comprises a 2MWT. A 2MWT may assess walking ability or functional capacity, or a combination of both. In aspects, an 2MWT may be tested using a timed task. In aspects, a 2MWT measures a distance walked by a subject in 2 minutes. In aspects, a 2MWT distance has increased post treatment with a composition provided herein. In aspects, a 2MWT may comprise assessing a subject in need thereof administered an anti-CD19 antibody or antigen-binding fragment thereof as compared to baseline. In aspects, a subject's distance has improved as compared to an otherwise comparable method wherein the subject of the otherwise comparable method is not administered an anti-CD19 antibody or antigen-binding fragment thereof.

In aspects, a 2MWT assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a 2MWT assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Short Form 12 (SF12) or 36 (SF36) Survey

In aspects, an assessment comprises a SF12 or SF36 survey. A SF12 or SF36 survey may assess a subject's quality of life. In aspects, an SF12 or SF36 survey is a self-reported survey. In aspects, a SF12 or SF36 survey assesses limitations in physical activities due to physical or emotional problems, limitations in social activities due to physical or emotional problems, limitations in usual role activities due to physical or emotional problems, bodily pain, general mental health, vitality (energy and fatigue), and general health perceptions. In aspects, a SF12 or SF36 survey is scored from 0 (worst health) to 100 (best health). In aspects, a SF12 or SF36 score has increased post treatment with a composition provided herein. In aspects, a SF12 or SF36 survey may comprise assessing a subject in need thereof administered an anti-CD19 antibody or antigen-binding fragment thereof as compared to baseline. In aspects, a subject's score has improved as compared to an otherwise comparable method wherein the subject of the otherwise comparable method is not administered an anti-CD19 antibody or antigen-binding fragment thereof.

In aspects, a SF12 or SF36 assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a SF12 or SF36 assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Patient-Reported Outcomes Measurement Information System (PROMIS) Short Form 7a (Fatigue)

In aspects, an assessment comprises a PROMIS fatigue 7a survey. A PROMIS fatigue 7a survey may assess a range of self-reported symptoms, from mild subjective feelings of tiredness to an overwhelming, debilitating, and sustained sense of exhaustion. In aspects, fatigue is divided into the experience of fatigue (frequency, duration, and intensity) and the impact of fatigue on physical, mental, and social activities. In aspects, a PROMIS fatigue 7a survey assesses symptoms over the past week. In aspects, a PROMIS fatigue 7a survey may comprise assessing a subject in need thereof administered an anti-CD19 antibody or antigen-binding fragment thereof as compared to baseline. In aspects, a subject's self-reported symptoms are improved as compared to an otherwise comparable method wherein the subject of the otherwise comparable method is not administered an anti-CD19 antibody or antigen-binding fragment thereof.

In aspects, a PROMIS fatigue 7a assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PROMIS fatigue 7a assessment to an anti-CD19 antibody or antigen-binding fragment thereof can be assessed on about day 1, week 12±7 days, week 26±3 days, week 34±7 days, week 38±7 days, and week 46±7 days post treatment initiation.

### PROMIS Short Form 8a

In aspects, an assessment comprises a PROMIS pain interference 8a survey. A PROMIS pain interference 8a survey may assess the consequences of pain on a subject's life. A PROMIS pain interference 8a survey may include the extent to which pain hinders engagement with social, cognitive, emotional, physical, and recreational activities. In aspects, a PROMIS pain interference 8a survey may comprise assessing a subject in need thereof administered an anti-CD19 antibody or antigen-binding fragment thereof as compared to baseline. In aspects, a subject's pain hindrance is improved as compared to an otherwise comparable method wherein the subject of the otherwise comparable method is not administered an anti-CD19 antibody or antigen-binding fragment thereof.

In aspects, a PROMIS short form 8a assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PROMIS short form 8a assessment to an anti-CD19 antibody or antigen-binding fragment thereof can be assessed on about day 1, week 12±7 days, week 26±3 days, week 34±7 days, week 38±7 days, and week 46±7 days post treatment initiation.

### PROMIS Short Form 8b

In aspects, an assessment comprises a PROMIS physical function 8b survey. A PROMIS physical function 8b survey may assess a range of self-reported capability. A PROMIS physical function 8b survey may include the functioning of a subject's upper extremities (e.g., dexterity), lower extremities (e.g., walking or mobility), and central regions (e.g., neck and back), as well as daily activities (e.g., running errands). In aspects, a PROMIS physical function 8b survey may comprise assessing a subject in need thereof administered an anti-CD19 antibody or antigen-binding fragment thereof as compared to baseline. In aspects, a subject's self-reported capability is improved as compared to an otherwise comparable method wherein the subject of the otherwise comparable method is not administered an anti-CD19 antibody or antigen-binding fragment thereof.

In aspects, a PROMIS physical function 8b assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PROMIS physical function 8b assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Handheld Dynamometry

In aspects, an assessment comprises handheld dynamometry. In aspects, handheld dynamometry may assess strong reliability, construct validity, and responsiveness in myositis patients (Saygin et al, 2021), especially to evaluate strength of single muscle groups (Baschung et al, 2018). Handheld dynamometry may be used to evaluate muscle strength. In aspects, a handheld dynamometry assessment may evaluate strength of a single muscle group at a time. In aspects, a handheld dynamometry assessment for a subject administered an anti-CD19 antibody or antigen-binding fragment thereof is determined as compared to baseline. In aspects, a subject's score is improved as compared to an otherwise comparable method wherein the subject of the otherwise comparable method is not administered an anti-CD19 antibody or antigen-binding fragment thereof.

In aspects, a handheld dynamometry assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a handheld dynamometry assessment to an anti-CD19 antibody or antigen-binding fragment thereof can be assessed on about day 1, week 12±7 days, week 26±3 days, week 34±7 days, week 38±7 days, and week 46±7 days post treatment initiation.

Patient Global Impression of Severity (PGIS) survey

In aspects, an assessment comprises a PGIS survey. A PGIS survey may use a categorical scale to ask a subject how much their myopathy has changed. In aspects, a PGIS survey may be administered after a subject in need thereof has been administered an anti-CD19 antibody or antigen-binding fragment thereof of the disclosure. In aspects, a PGIS survey score is at least about 1, about 2, about 3, about 4, or about 5. In aspects, a PGIS survey scale may range from 1 (much better) to 5 (much worse). In aspects, a level of a PGIS survey is reduced by at least about 1, about 2, about 3, or about 4 points post treatment with a composition provided herein. In aspects, a PGIS survey may comprise assessing a subject in need thereof administered an anti-CD19 antibody or antigen-binding fragment thereof as compared to baseline. In aspects, a subject's score is reduced as compared to an otherwise comparable method wherein the subject of the otherwise comparable method is not administered an anti-CD19 antibody or antigen-binding fragment thereof.

In aspects, a PGIS assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PGIS assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Patient Global Impression of Change (PGIC) Survey

In aspects, an assessment comprises a PGIC survey. A PGIC survey may use a categorical scale to ask a subject how much their myopathy has changed. In aspects, a PGIC survey may be administered after a subject in need thereof has been administered an anti-CD19 antibody or antigen-binding fragment thereof of the disclosure. In aspects, a PGIC survey score is at least about 1, about 2, about 3, about 4, or about 5. In aspects, a PGIC survey scale may range from 1 (much better) to 5 (much worse). In aspects, a level of a PGIC survey is reduced by at least about 1, about 2, about 3, or about 4 points post treatment with a composition provided herein. In aspects, a PGIC survey may comprise assessing a subject in need thereof administered an anti-CD19 antibody or antigen-binding fragment thereof as compared to baseline. In aspects, a subject's score is reduced as compared to an otherwise comparable method wherein the subject of the otherwise comparable method is not administered an anti-CD19 antibody or antigen-binding fragment thereof.

In aspects, a PGIC assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PGIC assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Clinical Global Impression (CGIC) Survey

In aspects, an assessment comprises a CGIC survey. A CGIC survey may use a categorical scale to determine how much a subject health has changed. In aspects, a CGIC survey may assess a subject's cognitive, function, or behavioral conditions, or combinations thereof. In aspects, a CGIC survey may be administered to a clinician monitoring a subject in need thereof who has been administered an anti-CD19 antibody or antigen-binding fragment thereof of the disclosure. In aspects, a CGIC survey score is at least about 1, about 2, about 3, about 4, about 5, about 6, or about 7. In aspects, a CGIC survey scale may range from 1 (normal) to 7 (among the most ill patients). In aspects, a level of a CGIC survey is reduced by at least about 1, about 2, about 3, about 4, about 5, or about 6 points post treatment with a composition provided herein. In aspects, a CGIC survey may comprise assessing a clinician monitoring a subject in need thereof administered an anti-CD19 antibody or antigen-binding fragment thereof as compared to baseline. In aspects, a clinicians score of a subject is reduced as compared to an otherwise comparable method wherein the subject of the otherwise comparable method is not administered an anti-CD19 antibody or antigen-binding fragment thereof.

In aspects, a CGIC assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a CGIC assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Myositis Functional Index (FI3) Survey

In aspects, an assessment comprises a myositis FI3 survey. A myositis FI3 survey may be used to assess the functional outcome of muscle endurance and function of frequently affected muscle groups (MFI3 2016). In aspects, a myositis FI3 assessment is scored from about 1, 2, 3, 4, 5, 6, 7, 8, 9, up to about 10. In aspects, myositis FI3 assessment is scored from about 0-10, about 1-5, about 4-10, or about 1-10. In aspects, a myositis FI3 score ranges from 0 (no exertion) to 10 (maximal exertion). In aspects, a myositis FI3 assessment is scored as the number of correctly performed repetitions with 60 or 120 maximal number of repetitions depending on the muscle group assessed. In aspects, a myositis FI3 assessment may comprise assessing a subject in need thereof administered an anti-CD19 antibody or antigen-binding fragment thereof as compared to baseline. In aspects, a subject's myositis FI3 score is reduced as compared to an otherwise comparable method wherein the subject of the otherwise comparable method is not administered an anti-CD19 antibody or antigen-binding fragment thereof.

In aspects, a myositis FI3 assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a myositis FI3 assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Global Pain 24-Hour Numeric Rating Scale (NRS)

In aspects, an assessment comprises a global pain 24-hour NRS survey. A global pain 24-hour NRS survey may assess the intensity of pain a subject may have experienced in the past 24 hours. In aspects, global pain 24-hour NRS survey may be scored from about 1, 2, 3, 4, 5, 6, 7, 8, 9, and up to about 10. In aspects, a global pain 24-hour NRS survey is from about 0-5, about 0-10, or about 1-10. A global pain 24-hour NRS survey scale may range from 0 (no pain) to 10 (the most intense pain imaginable). In aspects, a global pain 24-hour NRS survey may comprise assessing a subject in need thereof administered an anti-CD19 antibody or antigen-binding fragment thereof as compared to baseline. In aspects, a subject's score is reduced as compared to an otherwise comparable method wherein the subject of the otherwise comparable method is not administered an anti-CD19 antibody or antigen-binding fragment thereof.

In aspects, a global pain 24-hour NRS assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a global pain 24-hour NRS assessment to an anti-CD19 antibody or antigen-binding fragment thereof can be assessed on about day 1, week 12±7 days, week 26±3 days, week 34±7 days, week 38±7 days, and week 46±7 days post treatment initiation.

5-level 5Q-5D (EQ-5D-5L)

In aspects, an assessment comprises an EQ-5D-5L survey. An EQ-5D-5L survey may be used to assess mobility, self-care, usual activities, pain-discomfort, or anxiety/depression, and combinations thereof. In aspects, an EQ-5D-5L survey may be administered to a subject in need thereof who has been administered an anti-CD19 antibody or antigen-binding fragment thereof of the disclosure. In aspects, an EQ-5D-5L survey is scored using a visual analog scale (VAS). In aspects, an EQ-5D-5L survey score is at least about 1, about 2, about 3, about 4, or about 5. In aspects, an EQ-5D-5L survey scale may range from 1 (best) to 5 (worst). In aspects, a level of an EQ-5D-5L survey is reduced by at least about 1, about 2, about 3, or about 4 points post treatment with a composition provided herein. In aspects, an EQ-5D-5L survey may comprise assessing a subject in need thereof administered an anti-CD19 antibody or antigen-binding fragment thereof as compared to baseline. In aspects, a subject's score is reduced as compared to an otherwise comparable method wherein the subject of the otherwise comparable method is not administered an anti-CD19 antibody or antigen-binding fragment thereof.

In aspects, an EQ-5D-5L assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, an EQ-5D-5L assessment to an anti-CD19 antibody or antigen-binding fragment thereof can be assessed on about day 1, week 12±7 days, week 26±3 days, week 34±7 days, week 38±7 days, and week 46±7 days post treatment initiation.

### Muscle-Associated Enzymes

In aspects, an assessment comprises measuring a level of a muscle-associated enzyme. Muscle-associated enzymes assessed may include, but are not limited to, alanine transaminase (ALT), aspartate transaminase (AST), lactate dehydrogenase (LDH), creatine kinase (CK), and aldolase, and combinations thereof. In aspects, CK is assessed. In aspects, whole blood, plasma, serum, and urine are collected to assessed levels of muscle-associated enzymes. In aspects, assessing a level of a muscle-associated enzyme may comprise assessing a subject in need thereof administered an anti-CD19 antibody or antigen-binding fragment thereof as compared to baseline. In aspects, administration of an anti-CD19 antibody or antigen-binding fragment thereof of the disclosure is effective in reducing a level of a muscle-associated enzyme in a subject by at least about or at most about 10%, 20%, 30%, 40%, 50%, 55%, 60%, 65%, 70%, 75%, 80%, 85%, 90%, 95%, 96%, 97%, 98%, 99%, or up to about 100% of the level prior the administration. In aspects, administration of an anti-CD19 antibody or antigen-binding fragment thereof of the disclosure is effective in reducing a level of a muscle-associated enzyme in a subject by at least about or at most about: 3%-5%, 5%-10%, 10%-20%, 5%-25%, 20-40%, 30-50%, 40-60%, 45-75%, 60-80%, 75-95%, or 80-100% as compared to a baseline level prior to the administration.

In aspects, a muscle-associated enzyme assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a muscle-associated enzyme assessment to an anti-CD19 antibody or antigen-binding fragment thereof can be assessed on about day-28, day −27, day −26, day −25, day −24, day −23, day −22, day −21, day −20, day −19, day −18, day −17, day −16, day −15, day −14, day −13, day −12, day −11, day −10, day −9, day −8, day −7, day −6, day −5, day −4, day −3, day −2, day −1, day 0, day 1, week 2±3 days, week 6±7 days, week 12±7 days, week 18±7 days, week 22±7 days, week 26±3 days, week 30±3 days, week 34±7 days, week 38±7 days, week 42±7 days, week 46±7 days, and week 52±7 days post treatment initiation.

### Pharmacokinetic Assessment

In aspects, a method provided herein can comprise determining a concentration of an anti-CD19 antibody or antigen-binding fragment thereof in a subject in need thereof post administration. In aspects, a method comprises a pharmacokinetic assessment. In aspects, a sample is a blood sample or a plasma sample, or a combination of both. In aspects, a suitable assay to measure pharmacokinetics may comprise electrochemiluminescence (ECL) assay, a bead-based assay, a cell-based assay, and combinations thereof. In aspects, a sample may comprise plasma and the plasma is assessed for an anti-CD19 antibody or antigen-binding fragment thereof concentration by measuring: maximum observed concentration (Cₘₐₓ), area under the concentration-time curve (AUC), clearance (CL), and terminal elimination half-life (t1/2).

In aspects, a pharmacokinetic assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a pharmacokinetic assessment to an anti-CD19 antibody or antigen-binding fragment thereof can be assessed on about day −28, day −27, day −26, day −25, day −24, day −23, day −22, day −21, day −20, day −19, day −18, day −17, day −16, day −15, day −14, day −13, day −12, day −11, day −10, day −9, day −8, day −7, day −6, day −5, day −4, day −3, day −2, day −1, day 0, day 1, week 2±3 days, week 6±7 days, week 12±7 days, week 18±7 days, week 26±3 days, week 30±3 days, week 34±7 days, week 38±7 days, and week 52±7 days post treatment initiation.

### Immunogenicity

In aspects, an assessment comprises determining a level of immunogenicity, if any, of an anti-CD19 antibody or antigen-binding fragment thereof of the disclosure. Immunogenicity comprises determining the status and/or titer of anti-drug antibodies (ADA) to an anti-CD19 antibody or antigen-binding fragment thereof. The presence of ADA can be evaluated using a plasma sample from a subject administered an anti-CD19 antibody or antigen-binding fragment thereof. In aspects, ADA are not detected post administration of an anti-CD19 antibody or antigen-binding fragment thereof. In aspects, ADA levels are reduced as compared to an otherwise comparable method wherein the subject of the otherwise comparable method is not administered an anti-CD19 antibody or antigen-binding fragment thereof; for example, the reduction may be about: 10%, 15%, 20%, 25%, 50%, 60%, 70%, 80%, 90%, 95%, 96%, 97%, 98%, 99%, or up to 100% as compared to ADA levels in an otherwise comparable method wherein the subject of the otherwise comparable method is not administered an anti-CD19 antibody or antigen-binding fragment thereof.

In aspects, a immunogenicity assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a immunogenicity assessment to an anti-CD19 antibody or antigen-binding fragment thereof can be assessed on about day −28, day −27, day −26, day −25, day −24, day −23, day −22, day −21, day −20, day −19, day −18, day −17, day −16, day −15, day −14, day −13, day −12, day −11, day −10, day −9, day −8, day −7, day −6, day −5, day −4, day −3, day −2, day −1, day 0, day 1, week 2±3 days, week 6±7 days, week 12±7 days, week 18±7 days, week 26±3 days, week 30±3 days, week 34±7 days, week 38±7 days, and week 52±7 days post treatment initiation.

### Transcriptomics

In aspects, an assessment comprises quantifying expression levels of genes or gene pathways associated with disease activity by way of whole blood transcriptomics. In aspects, whole blood transcriptomics may be assessed via RNA isolation from whole blood. In aspects, RNA testing may be performed to assess changes over time in the expression of genes or gene pathways associated with myositis, mechanism of action of or in response to an anti-CD19 antibody or antigen-binding fragment thereof administration, or the pathogenesis of myositis.

RNA may be isolated at baseline from whole blood to test for changes in expression levels of the genes or gene pathways associated with disease activity. In aspects, transcriptome profiling is assessed by methods including, but not limited to, qPCR, RNAseq, and exome sequencing.

In aspects, expression levels of genes associated with disease activity by way of RNA analysis may be reduced by at least about 2-fold, 3-fold, 4-fold, 5-fold, 10-fold, 20-fold, 40-fold, 60-fold, 80-fold, 100-fold, 120-fold, 140-fold, 160-fold, 180-fold, 200-fold, 220-fold, 240-fold, 260-fold, 280-fold, or up to about 300-fold post administration as compared to an otherwise comparable method wherein the subject of the otherwise comparable method is not administered an anti-CD19 antibody or antigen-binding fragment thereof. In aspects, expression levels of genes associated with disease activity by way of RNA analysis may be reduced by at least about 10%, 15%, 20%, 25%, 50%, 60%, 70%, 80%, 90%, 95%, 96%, 97%, 98%, 99%, or up to 100% as compared to an otherwise comparable method wherein the subject of the otherwise comparable method is not administered an anti-CD19 antibody or antigen-binding fragment thereof.

In aspects, a transcriptomics analysis to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a transcriptomics analysis to an anti-CD19 antibody or antigen-binding fragment thereof can be assessed on about day 1, week 6±7 days, week 12±7 days, week 26±3 days, week 30±7, week 34±7, week 38±7 week 46±7 days, week 52±7 days, week 78 weeks±7 days, week 104±7 days, and week 116±7 days post treatment initiation.

### Whole Blood for DNA, SNP, Epigenetic Analysis

In aspects, an assessment comprises determining the sequence of genes associated with disease activity by way of whole blood DNA analysis. In aspects, DNA testing may be performed to assess changes over time in the sequence of genes, including: single nucleotide polymorphisms (SNPs) associated with the development of myositis (e.g., IMNM) genes that may be related to the mechanism of action of an anti-CD19 antibody or antigen-binding fragment thereof.

In aspects, whole blood can be collected and may be used to evaluate gene sequences before, during, and after treatment with any of the compositions provided herein. Gene sequences found to be modulated by treatment may be analyzed in whole blood using quantitative methods. Samples may be used to examine gene sequences and their changes over time as assessed by NGS, Sanger Sequencing, or PCR.

In aspects, determining changes in gene sequences associated with disease activity and/or treatment efficacy by way of DNA analysis to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, determining changes in gene sequences associated with disease activity and/or treatment efficacy by way of DNA analysis to an anti-CD19 antibody or antigen-binding fragment thereof can be assessed on about day 1, week 6±7 days, week 12±7 days, week 26±3 days, week 38±7 week 46±7 days, week 52±7 days, week 78 weeks±7 days, week 104±7 days, and week 116±7 days post treatment initiation.

### Pharmacodynamic Assessment

In aspects, a method comprises a pharmacodynamic assessment. In aspects, an assessment can occur over a period of time. In aspects, a sample comprises a blood sample, serum sample, plasma sample, or muscle sample. In aspects, a muscle biopsy location may be identified by MRI. In aspects, a muscle biopsy may be taken via non-invasive surgery (e.g., biopsy needle) or invasive surgery (i.e., open biopsy). Suitable assays to assess a biomarker level may comprise flow cytometry, histology, immunohistochemistry, blood analysis, microscopy, PCR, ELISA, and combinations thereof.

In aspects, a method provided herein can comprise determining a level of a biomarker including, but not limited to, SPR, HMGCR, total Ig, IgA, IgM, IgG, CD19+ cells (e.g., CD19+ B cells), CD20+ cells (e.g., CD20-plasmablasts, CD20-plasma cells), and peripheral blood mononuclear cells (PBMCs, e.g., T-cells, B-cells), post administration of an anti-CD19 antibody or antigen-binding fragment thereof to a subject in need thereof. Exemplary B cells include, but are not limited to, circulating B cells, blood B cells, splenic B cells, marginal zone B cells, follicular B cells, peritoneal B cells, bone marrow B cells, progenitor B cells, early pro-B cells, late pro-B cells, large-pre-B cells, small pre-B cells, immature B cells, mature B cells, antigen stimulated B cells, and/or plasma cells. In aspects, a reduction of biomarkers may be detected as compared to an otherwise comparable method wherein the subject of the otherwise comparable method is not administered an anti-CD19 antibody or antigen-binding fragment thereof. In aspects, elimination of a biomarker may be detected as compared to an otherwise comparable method wherein the subject of the otherwise comparable method is not administered an anti-CD19 antibody or antigen-binding fragment thereof as compared to an otherwise comparable method lacking the administering of an anti-CD19 antibody or antigen-binding fragment thereof. In aspects, reduction of a biomarker comprises at least about or at most about: 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 105-fold, 110-fold, 115-fold, 120-fold, 125-fold, 130-fold, 135-fold, 140-fold, 145-fold, 150-fold, 155-fold, 160-fold, 165-fold, 170-fold, 175-fold, 180-fold, 185-fold, 19-fold, 195-fold, 200-fold, 210-fol, 220-fold, 230-fold, 240-fold, 250-fold, 260-fold, 270-fold, 280-fold, 290-fold, or up to about 300-fold reduction as compared to an otherwise comparable method lacking the administering. In aspects, treatment efficacy of an anti-CD19 antibody or antigen-binding fragment thereof on a biomarker can be assessed over time using a suitable immunoassay. In aspects, effects of an anti-CD19 antibody or antigen-binding fragment thereof are assessed over time using a qualified immunoassay.

The disclosure provides for an anti-CD19 antibody containing compositions that efficiently reduce or deplete B cells in a subject. Because an anti-CD19 antibody or antigen-binding fragment thereof binds to and depletes a biomarker, the reduction or elimination can be used as a measure of treatment efficacy. In aspects, total B cells is a measure of target engagement.

In aspects, an anti-CD19 antibody or antigen-binding fragment thereof may achieve at least about 10%, 15%, 20%, 25%, 50%, 60%, 70%, 80%, 90%, 95%, 96%, 97%, 98%, 99%, or up to about 100% reduction in B cells levels as compared to an otherwise comparable method wherein the subject of the otherwise comparable method is not administered an anti-CD19 antibody or antigen-binding fragment thereof. Reduction or elimination of SPR, HMGCR, total Ig, IgA, IgM, IgG, CD19+ cells (e.g., CD19+ B cells), CD20+ cells (e.g., CD20-plasmablasts, CD20-plasma cells), and peripheral blood mononuclear cells (PBMCs, e.g., B cells), or a combination thereof, may persist for extended periods of time. In aspects, depletion of SPR, HMGCR, total Ig, IgA, IgM, IgG, CD19+ cells (e.g., CD19+ B cells), CD20+ cells (e.g., CD20-plasmablasts, CD20-plasma cells), and peripheral blood mononuclear cells (PBMCs, e.g., B cells), or a combination thereof, may persist for at least 1 day, at least 2 days, at least 3 days, at least 4 days, at least 5 days, at least 6 days, at least 7 days, at least 8 days, at least 9 days, at least 10 days, at least 15 days, at least 20 days, at least 25 days, or at least 30 days. In aspects, depletion of SPR, HMGCR, total Ig, IgA, IgM, IgG, CD19+ cells (e.g., CD19+ B cells), CD20+ cells (e.g., CD20-plasmablasts, CD20-plasma cells), and peripheral blood mononuclear cells (PBMCs, e.g., B cells), or a combination thereof, may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In aspects, depletion of SPR, HMGCR, total Ig, IgA, IgM, IgG, CD19+ cells (e.g., CD19+ B cells), CD20+ cells (e.g., CD20-plasmablasts, CD20-plasma cells), and peripheral blood mononuclear cells (PBMCs, e.g., B cells), or a combination thereof, may persist for at least 1 month, at least 2 months, at least 3 months, at least 4 months, at least 5 months, at least 6 months, at least 7 months, at least 8 months, at least 9 months, at least 10 months, at least 11 months, or at least 12 months.

In aspects, a pharmacodynamic assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a pharmacodynamic assessment to an anti-CD19 antibody or antigen-binding fragment thereof can be assessed on about day −28, day −27, day −26, day −25, day −24, day −23, day −22, day −21, day −20, day −19, day −18, day −17, day −16, day −15, day −14, day −13, day −12, day −11, day −10, day −9, day −8, day −7, day −6, day −5, day −4, day −3, day −2, day −1, day 0, day 1, week 2±3, week 6±7 days, week 12±7 days, week 18±7 days, week 22±7 days, week 26±3 days, week 30±3 days, week 34±7 days, week 38±7 days, week 42±7 days, week 46±7 days, week 52±7 days, week 78±7 days, week 104±7 days, and week 116±7 days post treatment initiation.

### Eligibility for Open-Label Period (OLP)

In aspects, an assessment comprises determining eligibility for a subject to enroll in OLP, also known as an open label extension (OLE). In aspects, an assessment comprise determining whether a subject in need thereof having previously been administered an anti-CD19 antigen-binding fragment thereof may include: completed Phase 2 or Phase 3, discontinued any non-B cell-depleting/non-GC immunomodulatory agent, or planned tapering of any GC, or combinations thereof.

In aspects, an assessment determining eligibility for a subject to enroll in OLP can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation.

In aspects, an assessment determining eligibility for a subject to enroll in OLP can be assessed on about day 1, week 26±3 days, and week 52±7 days post treatment initiation.

### Health Assessment Questionnaire-Disability Index (HAQ-DI)

In aspects, an assessment comprises HAQ-DI. HAQ-DI assesses physical function of a subject in need thereof (HAQ-DI Form 05a). In aspects, HAQ-DI may be scored from about 0, 1, 2, or up to about 3. In aspects, a HAQ-DI score is at least about 0, about 1, about 2, or about 3. In aspects, HAQ-DI may comprise 8 sections: dressing and grooming, arising, eating, walking, hygiene, reach, grip, and common daily activities. In aspects, each section is scored from 0 (without any difficulty) to 3 (unable to do). In aspects, a HAQ-DI assessment may comprise assessing a subject in need thereof administered an anti-CD19 antibody or antigen-binding fragment thereof as compared to baseline. In aspects, a subject's HAQ-DI score is reduced as compared to an otherwise comparable method wherein the subject of the otherwise comparable method is not administered an anti-CD19 antibody or antigen-binding fragment thereof.

In aspects, a HAQ-DI assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a HAQ-DI assessment to an anti-CD19 antibody or antigen-binding fragment thereof can be assessed on about week 26±3 days and week 52±7 days post treatment initiation.

### Patient Global Assessment of Disease Activity (PGA)

In aspects, an assessment comprises patient global assessment of disease activity (PGA). PGA measures the global evaluation by the patient of the patient's overall disease activity at the time of assessment. In aspects, a PGA assessment is scored from about 1, 2, 3, 4, 5, 6, 7, 8, 9, up to about 10. In aspects, PGA assessment is scored from about 0-10, about 1-5, about 4-10, or about 1-10. In aspects, PGA may be measured using a 10 cm visual analog scale (VAS) that ranges from “no evidence of disease activity” to “extreme active or severe disease activity.” In aspects, a PGA assessment may comprise assessing a subject in need thereof administered an anti-CD19 antibody or antigen-binding fragment thereof as compared to baseline. In aspects, a subject's PGA score is reduced as compared to an otherwise comparable method wherein the subject of the otherwise comparable method is not administered an anti-CD19 antibody or antigen-binding fragment thereof.

In aspects, a PGA assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a PGA assessment to an anti-CD19 antibody or antigen-binding fragment thereof can be assessed on about week 26±3 days and week 52±7 days post treatment initiation.

### Composite Glucocorticoid Toxicity Index (CGTI)

In aspects, an assessment comprises a CGTI survey. In aspects, a CGTI survey assesses glucocorticoid (GC)-related treatment morbidity and GC-sparing ability post treatment initiation. In aspects, a CGTI survey may comprise assessing: body mass index (BMI), glucose tolerance, blood pressure, lipids, bone density, steroid myopathy, skin toxicity, neuropsychiatric toxicity, infection, gastrointestinal, musculoskeletal, and ocular. In aspects, a subject in need thereof having been administered a CD19 antibody or antigen-binding fragment thereof is assessed for CGTI. In aspects, a CGTI is scored from about −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, or up to about 439. In aspects, a CGTI survey may comprise assessing a subject in need thereof administered an anti-CD19 antibody or antigen-binding fragment thereof as compared to baseline. In aspects, a subject's CGTI score is reduced as compared to an otherwise comparable method wherein the subject of the otherwise comparable method is not administered an anti-CD19 antibody or antigen-binding fragment thereof.

In aspects, a CGTI assessment to an anti-CD19 antibody or antigen-binding fragment thereof can be assessed on about −28 days, −27 days, −26 days, −25 days, −24 days, −23 days, −22 days, −21 days, −20 days, −19 days, −18 days, −17 days, −16 days, −15 days, −14 days, −13 days, −12 days, −11 days, −10 days, −9 days, −8 days, −7 days, −6 days, −5 days, −4 days, −3 days, −2 days, −1 day, 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 78 weeks, 104 weeks, or at least about 142 weeks post treatment initiation. In aspects, a CGTI assessment to an anti-CD19 antibody or antigen-binding fragment thereof can be assessed on about week 26±3 days and week 52±7 days post treatment initiation.

### Pharmaceutical Composition

In aspects, provided is a pharmaceutical composition. In aspects, provided are also therapeutic regiments comprising pharmaceutical compositions.

A pharmaceutical composition can comprise an anti-CD19 antibody or antigen-binding fragment thereof. In aspects, a pharmaceutical composition is part of a therapeutic regimen that comprises an anti-CD19 antibody or antigen-binding fragment thereof and one or more additional therapeutics provided herein. In aspects, the one or more additional therapeutics can comprise a corticosteroid, an anti-pain agent, or an immunomodulatory agent, or a combination thereof.

Many drugs can be administered orally as liquids, capsules, tablets, or chewable tablets. Because the oral route is the most convenient and usually the safest and least expensive, it is the one most often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver before being transported via the bloodstream to its target site. The intestinal wall and liver chemically alter (metabolize) many drugs, decreasing the amount of drug reaching the bloodstream. Consequently, these drugs are often given in smaller doses when injected intravenously to produce the same effect.

For a subcutaneous route, a needle is inserted into fatty tissue just beneath the skin. After a drug (e.g., an anti-CD19 antibody or antigen-binding fragment thereof) is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the abdomen, upper arm, thigh, or buttock. How quickly the drug is absorbed into the bloodstream depends, in part, on the blood supply to the muscle: the sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm.

In aspects, a pharmaceutical composition provided herein is administered via infusion. An infusion can take place over a period of time. For example, an infusion can be an administration of a pharmaceutical over a period of about 5 minutes to about 10 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, 5 hours, 6 hours, 7 hours, 8 hours, 9 hours, or up to about 10 hours. In aspects, intravenous administration is used to deliver a precise dose quickly and in a well-controlled manner throughout the body. It is also used for irritating solutions, which would cause pain and damage tissues if given by subcutaneous or intramuscular injection. When given intravenously, a drug is delivered immediately to the bloodstream and tends to take effect more quickly than when given by any other route. Prior to each IV infusion subjects may receive prophylaxis with IV methylprednisolone, oral diphenhydramine, and oral acetaminophen, or equivalent(s) to reduce the risk or severity of potential reactions. In aspects, a methylprednisolone dosage may range from about 10 mg to about 300 mg, about 20 mg to about 200 mg, about 30 mg to about 150 mg, about 40 mg to about 100 mg, about 50 mg to about 150 mg, or from about 75 mg to about 125 mg. In aspects, a methylprednisolone dose may be about 10 mg, 20 mg, 30 mg, 40 mg, 50 mg, 60 mg, 70 mg, 80 mg, 90 mg, 95 mg, 100 mg, 105 mg, 110 mg, 120 mg, 140 mg, 160 mg, 180 mg, 200 mg, 225 mg, 250 mg, 275 mg, or about 300 mg. In aspects, a methylprednisolone dosage may be about 100 mg. In aspects, an oral diphenhydramine dosage may range from about 5 mg to about 250 mg, about 10 mg to about 200 mg, about 15 mg to about 100 mg, about 20 mg to about 70, about 25 mg to about 50, about 15 mg to about 50, or about 25 mg to about 60 mg. In aspects, an oral diphenhydramine dosage may range from about 25 mg to about 50 mg. In aspects, an oral acetaminophen dosage may range from about 10 mg to about 1000 mg, from about 25 mg to about 750 mg, from about 50 mg to about 700 mg, from about 75 mg to about 650 mg, from about 100 mg to about 800 mg, from about 300 mg to about 900 mg, from about 400 mg to about 700 mg, or from about 500 mg to about 650 mg. In aspects, an oral acetaminophen dose may range from about 500 to about 650 mg.

In aspects, a pharmaceutical is administered intrathecally. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

Drugs administered by inhalation through the mouth can be atomized into smaller droplets than those administered by the nasal route, so that the drugs can pass through the windpipe (trachea) and into the lungs. How deeply into the lungs they go depends on the size of the droplets. Smaller droplets go deeper, which increases the amount of drug absorbed. Inside the lungs, they are absorbed into the bloodstream. Drugs applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In aspects, a treatment regime comprising a pharmaceutical composition may be dosed according to a body weight of a subject. In subjects who are determined obese (BMI>35) a practical weight may need to be utilized. In aspects, body surface area may be utilized to calculate a dosage.

In aspects, a pharmaceutical composition can be administered either alone or together with a pharmaceutically acceptable carrier or excipient, by any routes, and such administration can be carried out in both single and multiple dosages. More particularly, a pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, pharmaceutical formulations can be suitably sweetened and/or flavored by means of various agents of the type commonly employed for such purposes. Exemplary carriers and excipients can include dextrose, sodium chloride (NaCl), sucrose, lactose, cellulose, xylitol, sorbitol, maltitol, gelatin, PEG, PVP, histidine/histidine hydrochloride, trehalose dihydrate, polysorbate 80, and any combination thereof. In aspects, an excipient comprises: histidine/histidine hydrochloride, NaCl, trehalose dihydrate, and polysorbate 80.

Provided are various compositions, and methods of utilizing the same. In aspects, the compositions of the disclosure are used in methods of treatment of human subjects. In aspects, the subject in need thereof is pediatric (aged birth to 18 years). In aspects, the pediatric subject in need thereof is aged between about birth to about 5 years, about birth to about 4 years, about 3 years to about 10 years, about 5 years to about 12 years, about 7 years to about 18 years, about 10 years to about 18 years, about 14 years to about 18 years, or about 16 years to about 18 years. In aspects, the pediatric subject is aged at least about 16 years. In aspects, the pediatric subject is aged about 16 years to about 18 years. In aspects, the pediatric subject is about 10-18, 12-18, 14-18, 16-18, 17-18, or newborn-18.

### NUMBERED EMBODIMENTS

Notwithstanding the appended claims, the following numbered embodiments also form part of the instant disclosure.

### Embodiment Set A

1. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8.
2. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, and wherein the subject in need thereof is pediatric.
3. The method of embodiment 2, wherein the pediatric subject is 16-18 years old.
4. The method of any one of embodiments 1-3, wherein the subject is signal recognition particle-antibody positive (SRP-Ab+), 3-hydroxy-3-methylglutaryl-coenzyme A reductase-antibody positive (HMGCR-Ab+), or both SRP-Ab+ and HMGCR-Ab+.
5. The method of embodiment 4, wherein the subject is SRP-Ab+.
6. The method of embodiment 4, wherein the subject is HMGCR-Ab+.
7. The method of any one of embodiments 1-3, wherein the subject is signal recognition particle-antibody negative (SRP-Ab−), 3-hydroxy-3-methylglutaryl-coenzyme A reductase-antibody negative (HMGCR-Ab−), or both SRP-Ab− and HMGCR-Ab−.
8. The method of any one of embodiments 1-7, wherein the subject in need was previously treated with a conventional therapy.
9. The method of embodiment 8, wherein the conventional therapy comprises a corticosteroid, a non-steroidal immunosuppressant (IST), antibody therapy, or any combination thereof.
10. The method of embodiment 9, wherein the conventional therapy is a corticosteroid, and wherein the corticosteroid is prednisone.
11. The method of embodiment 9, wherein the conventional therapy is an IST selected from the group consisting of: methotrexate, cyclophosphamide, cyclosporine, azathioprine, mycophenolate mofetil, mycophenolic acid, tacrolimus, and any combination thereof.
12. The method of embodiment 9, wherein the conventional therapy is an antibody therapy is selected from the group consisting of: rituximab, ocrelizumab, obinutuzumab, ofatumumab, inebilizumab intravenous immunoglobulin (IVIg), and combinations thereof.
13. The method of any one of embodiments 8-12, wherein the conventional therapy is tapered following the administering of the effective amount of the anti-CD19 antibody or antigen-binding fragment thereof.
14. The method of embodiment 13, wherein the tapering comprises reducing a dosage of the conventional therapy every two weeks beginning on week 26.
15. The method of embodiment 13 or 14, wherein the conventional therapy comprises prednisone, and wherein the prednisone is tapered by 5 mg every two weeks following the administering.
16. The method of embodiment 15, wherein the tapering is continued at least until week 12.
17. The method of any one of embodiments 4-16, wherein the subject was determined to have at least one of SRP-Ab or HMGCR-Ab before the administering.
18. The method of any one of embodiments 1-17, wherein the administering of the anti-CD19 antibody or antigen-binding fragment thereof is repeated.
19. The method of any one of embodiments 1-17, wherein the administering is repeated on weeks 2, 26, 52, 78, 104, 130, and 156.
20. The method of any one of embodiments 1-19, wherein the administering, the repeated administering, or both are effective at reducing disease or symptoms thereof by week 12, 26, or 52.
21. The method of any one of embodiments 1-19, wherein the administering, the repeated administering, or both are effective at reducing disease or symptoms thereof by week 26 following the administering as determined by an increased 2016 ACR/EULAR total improvement score (TIS) as compared to a baseline score of the subject or a score in an untreated subject.
22. The method of any one of embodiments 1-19, wherein the administering, the repeated administering, or both are effective at reducing disease or symptoms thereof by week 26 following the administering as compared to a baseline level of the subject or a level in an untreated subject.
23. The method of embodiment 22, wherein the reduced disease or symptoms thereof are determined by an improvement in muscle weakness, reduction in serum CK level, reduced atrophy, reduced edema, reduced fatty tissue replacement, reduced interstitial lung disease, reduced B cell levels, MMT8 score, and combinations thereof.
24. The method of any one of embodiments 1-23, wherein the subject is human.
25. The method of any one of embodiments 21-23, wherein the disease reduction is of at least about 3-fold, 5-fold, 20-fold, 40-fold, 60-fold, 80-fold, or up to about 100-fold.
26. The method of any one of embodiments 21-23, wherein the TIS score is ≥5, 10, 20, 30, 40, 50, 60, 80, or 100.
27. The method of any one of embodiments 1-26, wherein before the administering the subject in need thereof is prophylactically administered a glucocorticoid, an antihistamine, an anti-pain agent, and any combination thereof.
28. The method of embodiment 27, wherein the subject is prophylactically administered the glucocorticoid, the antihistamine, and the anti-pain agent.
29. The method of any one of embodiments 1-26, wherein the subject is prophylactically administered methylprednisolone, diphenhydramine, and acetaminophen.
30. The method of any one of embodiments 1-29, wherein the effective amount of the anti-CD19 antibody or antigen-binding fragment thereof is from about: 50 mg to about 1000 mg, 100-800 mg, 200-600 mg, 200-500 mg, 250-450 mg, 200-350 mg, 250-350 mg, or 375-325 mg.
31. The method of any one of embodiments 1-29, wherein the effective amount is from about: 50 mg, 100 mg, 200 mg, 225 mg, 250 mg, 275 mg, 300 mg, 325 mg, 350 mg, 375 mg, 400 mg, 500 mg, 600 mg, 700 mg, 800 mg, 900 mg, or 1000 mg.
32. The method of embodiment 31, wherein the effective amount is about 300 mg.
33. The method of any one of embodiments 1-32, wherein the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively.
33. The method of any one of embodiments 1-32, wherein the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.
34. The method of any one of embodiments 1-33, wherein the anti-CD19 antibody is a monoclonal antibody.
35. The method of any one of embodiments 1-34, wherein the anti-CD19 antibody is afucosylated.
36. The method of any one of embodiments 18-35, wherein the administering, the repeated administering, or both are effective at eliminating the IMNM for at least about 3 months, 6 months, 1 year, or 2 years.
37. The method of any one of embodiments 18-36, wherein the administering, the repeated administering, or both are effective at stabilizing or reducing the IMNM for at least about 3 months, 6 months, 1 year, or 2 years.
38. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is from about 100 mg-1000 mg.
39. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is about 300 mg.
40. A method of treatment, the method comprising administering an effective amount of an anti-CD19 antibody or antigen-binding fragment thereof to a pediatric subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively.
41. A method of treating Immune Mediated Necrotizing Myopathy (IMNM), the method comprising administering an effective amount of inebilizumab to a subject in need thereof, wherein the effective amount is about 100 mg-1000 mg.
42. A method of treatment, the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is positive for autoantibodies selected from the group consisting of: signal recognition particle (SRP), 3-hydroxy-3-methylglutaryl-coenzyme A reductase (HMGCR), and both SRP and HMGCR.
43. A method of treatment, the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is negative for autoantibodies selected from the group consisting of: signal recognition particle (SRP), 3-hydroxy-3-methylglutaryl-coenzyme A reductase (HMGCR), and both SRP and HMGCR.
44. The method of any one of embodiments 1-43, wherein the administering comprises 300 mg of the anti-CD19 antibody or antigen-binding fragment thereof on day 1 and day 15, followed by a single 300 mg infusion approximately every 6 months thereafter.

### Embodiment Set B

1. An anti-CD19 antibody or antigen-binding fragment thereof for use in the treatment of Immune Mediated Necrotizing Myopathy (IMNM), the anti-CD19 antibody or antigen-binding fragment thereof, comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8.
2. An anti-CD19 antibody or antigen-binding fragment thereof for use in the treatment of Immune Mediated Necrotizing Myopathy (IMNM) in a pediatric subject, the anti-CD19 antibody or antigen-binding fragment thereof, comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8.
3. The use of embodiment 2, wherein the pediatric subject is 16-18 years old.
4. The use of any one of embodiments 1-3, wherein the subject is signal recognition particle-antibody positive (SRP-Ab+), 3-hydroxy-3-methylglutaryl-coenzyme A reductase-antibody positive (HMGCR-Ab+), or both SRP-Ab+ and HMGCR-Ab+.
5. The use of embodiment 4, wherein the subject is SRP-Ab+.
6. The use of embodiment 4, wherein the subject is HMGCR-Ab+.
7. The use of any one of embodiments 1-3, wherein the subject is signal recognition particle-antibody negative (SRP-Ab−), 3-hydroxy-3-methylglutaryl-coenzyme A reductase-antibody negative (HMGCR-Ab−), or both SRP-Ab− and HMGCR-Ab−.
8. The use of any one of embodiments 1-7, wherein the subject in need was previously treated with a conventional therapy.
9. The use of embodiment 8, wherein the conventional therapy comprises a corticosteroid, a non-steroidal immunosuppressant (IST), antibody therapy, or any combination thereof.
10. The use of embodiment 9, wherein the conventional therapy is a corticosteroid, and wherein the corticosteroid is prednisone.
11. The use of embodiment 9, wherein the conventional therapy is an IST selected from the group consisting of: methotrexate, cyclophosphamide, cyclosporine, azathioprine, mycophenolate mofetil, mycophenolic acid, tacrolimus, and any combination thereof.
12. The use of embodiment 9, wherein the conventional therapy is an antibody therapy is selected from the group consisting of: rituximab, ocrelizumab, obinutuzumab, ofatumumab, inebilizumab intravenous immunoglobulin (IVIg), and combinations thereof.
13. The use of any one of embodiments 8-12, wherein the conventional therapy is tapered following the administering of the effective amount of the anti-CD19 antibody or antigen-binding fragment thereof.
14. The use of embodiment 13, wherein the tapering comprises reducing a dosage of the conventional therapy every two weeks beginning on week 26.
15. The use of embodiment 13 or 14, wherein the conventional therapy comprises prednisone, and wherein the prednisone is tapered by 5 mg every two weeks following the administering.
16. The use of embodiment 15, wherein the tapering is continued at least until week 12.
17. The use of any one of embodiments 4-16, wherein the subject was determined to have at least one of SRP-Ab or HMGCR-Ab before the administering.
18. The use of any one of embodiments 1-17, wherein the administering of the anti-CD19 antibody or antigen-binding fragment thereof is repeated.
19. The use of any one of embodiments 1-17, wherein the administering is repeated on weeks 2, 26, 52, 78, 104, 130, and 156.
20. The use of any one of embodiments 1-19, wherein the administering, the repeated administering, or both are effective at reducing disease or symptoms thereof by week 12, 26, or 52.
21. The use of any one of embodiments 1-19, wherein the administering, the repeated administering, or both are effective at reducing disease or symptoms thereof by week 26 following the administering as determined by an increased 2016 ACR/EULAR total improvement score (TIS) as compared to a baseline score of the subject or a score in an untreated subject.
22. The use of any one of embodiments 1-19, wherein the administering, the repeated administering, or both are effective at reducing disease or symptoms thereof by week 26 following the administering as compared to a baseline level of the subject or a level in an untreated subject.
23. The use of embodiment 22, wherein the reduced disease or symptoms thereof are determined by an improvement in muscle weakness, reduction in serum CK level, reduced atrophy, reduced edema, reduced fatty tissue replacement, reduced interstitial lung disease, reduced B cell levels, MMT8 score, and combinations thereof.
24. The use of any one of embodiments 1-23, wherein the subject is human.
25. The use of any one of embodiments 21-23, wherein the disease reduction is of at least about 3-fold, 5-fold, 20-fold, 40-fold, 60-fold, 80-fold, or up to about 100-fold.
26. The use of any one of embodiments 21-23, wherein the TIS score is ≥5, 10, 20, 30, 40, 50, 60, 80, or 100.
27. The use of any one of embodiments 1-26, wherein before the administering the subject in need thereof is prophylactically administered a glucocorticoid, an antihistamine, an anti-pain agent, and any combination thereof.
28. The use of embodiment 27, wherein the subject is prophylactically administered the glucocorticoid, the antihistamine, and the anti-pain agent.
29. The use of any one of embodiments 1-26, wherein the subject is prophylactically administered methylprednisolone, diphenhydramine, and acetaminophen.
30. The use of any one of embodiments 1-29, wherein the effective amount of the anti-CD19 antibody or antigen-binding fragment thereof is from about: 50 mg to about 1000 mg, 100-800 mg, 200-600 mg, 200-500 mg, 250-450 mg, 200-350 mg, 250-350 mg, or 375-325 mg.
31. The use of any one of embodiments 1-29, wherein the effective amount is from about: 50 mg, 100 mg, 200 mg, 225 mg, 250 mg, 275 mg, 300 mg, 325 mg, 350 mg, 375 mg, 400 mg, 500 mg, 600 mg, 700 mg, 800 mg, 900 mg, or 1000 mg.
32. The use of embodiment 31, wherein the effective amount is about 300 mg.
33. The use of any one of embodiments 1-32, wherein the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively.
34. The use of any one of embodiments 1-32, wherein the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.
35. The use of any one of embodiments 1-34, wherein the anti-CD19 antibody is a monoclonal antibody.
36. The use of any one of embodiments 1-35, wherein the anti-CD19 antibody is afucosylated.
37. The use of any one of embodiments 18-36, wherein the administering, the repeated administering, or both are effective at eliminating the IMNM for at least about 3 months, 6 months, 1 year, or 2 years.
38. The use of any one of embodiments 18-37, wherein the administering, the repeated administering, or both are effective at stabilizing or reducing the IMNM for at least about 3 months, 6 months, 1 year, or 2 years.
39. An anti-CD19 antibody or antigen-binding fragment thereof for use in the treatment of Immune Mediated Necrotizing Myopathy (IMNM), the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein from about 100 mg-1000 mg of the anti-CD19 antibody or antigen-binding fragment thereof is administered to a subject in need.
40. An anti-CD19 antibody or antigen-binding fragment thereof for use in the treatment of Immune Mediated Necrotizing Myopathy (IMNM), the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein about 300 mg of the anti-CD19 antibody or antigen-binding fragment thereof is administered to a subject in need.
41. An anti-CD19 antibody or antigen-binding fragment thereof for use in the treatment of a disease associated with CD19 expressing cells in a pediatric subject, the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively.
42. Inebilizumab for use in the treatment of Immune Mediated Necrotizing Myopathy (IMNM), wherein from about 100 mg-1000 mg of the inebilizumab is administered to a subject in need.
43. An anti-CD19 antibody or antigen-binding fragment thereof for use in the treatment of a disease associated with CD19 expressing cells, the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is positive for autoantibodies selected from the group consisting of: signal recognition particle (SRP), 3-hydroxy-3-methylglutaryl-coenzyme A reductase (HMGCR), and both SRP and HMGCR.
44. An anti-CD19 antibody or antigen-binding fragment thereof for use in the treatment of a disease associated with CD19 expressing cells, the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is negative for autoantibodies selected from the group consisting of: signal recognition particle (SRP), 3-hydroxy-3-methylglutaryl-coenzyme A reductase (HMGCR), and both SRP and HMGCR.
45. The use of any one of embodiments 1-44, wherein the administering comprises 300 mg of the anti-CD19 antibody or antigen-binding fragment thereof on day 1 and day 15, followed by a single 300 mg infusion approximately every 6 months thereafter.
46. An anti-CD19 antibody or antigen-binding fragment thereof for use in the treatment of Immune Mediated Necrotizing Myopathy (IMNM), the anti-CD19 antibody or antigen-binding fragment thereof comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein about 300 mg is administered every 6 months, and wherein two weeks prior to the first 300 mg administration every 6 months, an initial 300 mg dose is administered.
47. The use of any one of embodiments 38-46, wherein the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively.
48. The use of any one of embodiments 38-46, wherein the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.

### EXAMPLES

### Example 1—a Phase 2/3, Randomized, Double-Blind, Placebo-Controlled Study with an Open Label Extension to Evaluate the Efficacy and Safety of Inebilizumab in Adults and Adolescents≥16 Years Old with Immune-Mediated Necrotizing Myopathy

A phase 2/3, randomized, double-blind, placebo-controlled with an open label, efficacy and safety study of inebilizumab in adults and adolescents≥16 years old with immune-mediated necrotizing myopathy (IMNM) is disclosed herein.

### Objectives and Endpoints

Study objectives and endpoints are presented below and are the same for Phase 2 and Phase 3 unless otherwise specified, with an independent analysis of the two populations.

The primary objective is to assess whether inebilizumab can improve IMNM overall disease activity as measured by the total improvement score (TIS).

### Key Secondary Objective

To evaluate the safety and tolerability of inebilizumab in subjects with IMNM

To evaluate the effect of inebilizumab on other measures of disease activity

To characterize the pharmacokinetics (PK) and immunogenicity profile of inebilizumab in IMNM.

To assess whether inebilizumab can reduce the corticosteroid burden.

### Exploratory Objectives

To characterize the pharmacodynamic (PD) profile of inebilizumab and the effect on disease biomarkers.

To assess whether inebilizumab can improve IMNM overall disease activity as measured by muscle biopsy and MRI.

To assess the effect of inebilizumab on each antibody subtype.

To assess whether inebilizumab can prevent the use of rescue therapies or reduce the occurrence of clinical deterioration.

To assess the effect of inebilizumab on functional health and mobility in IMNM patients.

To assess the effect of inebilizumab on quality of life, healthcare resource utilization and well-being from the patient's perspective.

To assess whether inebilizumab can improve IMNM associated interstitial lung disease (ILD).

### Primary Endpoint

1. The primary efficacy endpoint for both studies is the mean 2016 ACR/EULAR total improvement score (TIS) at Week 26.

### Key Secondary Endpoints

1. Proportion of participants at Week 26 who improve ≥40 points on TIS and receive no rescue therapy after Week 8 during the randomized controlled period (RCP).
2. Proportion of participants at Week 26 with ≥8% improvement on MMT-8 and receive no rescue therapy after Week 8 during the RCP.
3. Median change from baseline in creatine kinase (CK) muscle levels at Week 26.
4. Proportion of participants who achieve key secondary endpoint 1, key secondary endpoint 2, and whose CK levels decrease by 50% from baseline at Week 26.

### Secondary Endpoints

1. Proportion of participants at Week 26 who improve ≥20 points on TIS and receive no rescue therapy after Week 8 during the RCP.
2. Proportion of participants at Week 26 who improve ≥30 points on TIS and receive no rescue therapy after Week 8 during the RCP.
3. Proportion of participants at Week 26 who improve ≥50 points on TIS and receive no rescue therapy after Week 8 during the RCP.
4. Proportion of participants at Week 26 who improve ≥60 points on TIS and receive no rescue therapy after Week 8 during the RCP.
5. Change from baseline in Patient Global Assessment of Disease Activity (PGA) at week 26 in Phase 2 and at week 52 in Phase 3.
6. Change from baseline in Health Assessment Questionnaire Disability Index (HAQ-DI) at week 26 in Phase 2 and at week 52 in Phase 3.
7. Change in steroid dose from baseline to last visit at week 26 in Phase 2 and at week 52 in Phase 3.
8. Proportion of participants who taper corticosteroid dose to 7.5 mg/day (or 50% reduction) and receive no rescue therapy after Week 8 during the RCP.
9. Proportion of participants who reach normalization or near normalization of CK levels and receive no rescue therapy after Week 8 during the RCP.
10. Proportion of participants at Week 26 with ≥10% improvement in MMT-8 score and receive no rescue therapy after Week 8 during the RCP.
11. Change from baseline in individual TIS core set measures at Week 26 in participants who receive no rescue therapy after Week 8 during the RCP.
12. Change from baseline in Proximal MMT at Week 26 in participants who receive no rescue therapy after Week 8 during the RCP.
13. Change from baseline in the Extramuscular Global Assessment at week 26 in Phase 2 and at week 52 in Phase 3.
14. Change from baseline in abbreviated handheld dynamometry at week 26 in Phase 2 and at week 52 in Phase 3.
15. Change from baseline in Patient Global Impression of Severity (PGI-S) at week 26 in Phase 2 and at week 52 in Phase 3.
16. Patient Global Impression of Change (PGI-C) at week 26 in Phase 2 and at week 52 in Phase 3.
17. Change from baseline in Clinical Global Impression of Severity (CGI-S) at week 26 in Phase 2 and at week 52 in Phase 3.
18. Change from baseline in Global Impression of Change (CGI-C) at week 26 in Phase 2 and at week 52 in Phase 3.
19. Change from baseline in the Myositis Functional Index (FI-3) at week 26 in Phase 2 and at week 52 in Phase 3.
20. Change from baseline in Patient-Reported Outcomes Information System (PROMIS) Pain Interference 8a v1.0 at week 26 in Phase 2 and at week 52 in Phase 3.
21. Change from baseline in pain numeric rating scale (NRS) assessing worst pain in the past 7 days at week 26 in Phase 2 and at week 52 in Phase 3.
22. Mean TIS at Week 26 for SRP-Ab+ and HMGCR-Ab+ subtypes independently.

### For Both Studies: Exploratory Endpoints

1. Change in anti-SRP and anti-HMGCR antibody titers.
2. Change in B and T cell flow cytometry (CD20+ B cell count) and immune cell subset phenotyping.
3. Change in exploratory endpoints related to disease or mechanism of action (blood gene expression profiles, serum/plasma biomarkers).
4. Effect of genetic markers on inebilizumab mechanism of action.
5. Proportion of participants who did not receive rescue therapy after Week 8 during the RCP.
6. Total instances of receipt of rescue therapy after Week 8.
7. Time to first protocol-defined clinical deterioration during RCP.
8. Change from baseline using disease-specific MRI results (optional substudy).
9. Change from baseline using pre- and post-treatment muscle biopsy (optional substudy).
10. Evaluate key secondary endpoints for SRP-Ab+ and HMGCR-Ab+ subtypes independently.
11. Change from baseline in extra muscular activity core set measure in SRP-Ab+ participants.
12. Change from baseline in PFT in participants with interstitial lung disease (ILD).
13. Change from baseline in PROMIS Short Form 7a (fatigue).
14. Change in Fatigue NRS assessing worst physical fatigue in the past 7 days [Time Frame: phase 2: up to 24 weeks; phase 3: up to 52 weeks].
15. Change from baseline in PROMIS Short Form 8a (pain interference).
16. Change from baseline in PROMIS Short Form 8b (physical function).
17. Change from baseline in Short Form-12.
18. Change in the Physical Functioning subscale and Physical Component Summary scores of the 36-Item Short Form Survey version 2 (SF-36v2) at week 26 in Phase 2 and at week 52 in Phase 3.
19. Composite Glucocorticoid Toxicity Index (C-GTI) at week 26 in Phase 2 and at week 52 in Phase 3.
20. Change from baseline in EQ-5D-5L utilities at week 26 in Phase 2 and at week 52 in Phase 3.
21. Change from baseline in EQ-5D-5L VAS at week 26 in Phase 2 and at week 52 in Phase 3.
22. Change from baseline in timed get up and go test at week 26 in Phase 2 and at week 52 in Phase 3.
23. Change from baseline in 2-minute walk test at week 26 in Phase 2 and at week 52 in Phase 3.

### Both Studies: Other Endpoints

1. Pharmacokinetic (PK) profile of inebilizumab over time.
2. Anti-drug antibody (ADA) status and titer over time.

### Study Design

This study is a phase 2/3, randomized, double-blind, placebo-controlled study. The target population is adult subjects aged ≥16 years with signal recognition particle-antibody positive (SRP-Ab+) or 3-hydroxy-3-methylglutaryl-coenzyme A reductase-antibody positive (HMGCR-Ab+) immune-mediated necrotizing myopathy (IMNM). Under this single study are two separate phases. In each stage, a subject who completes their respective randomized control phase of their trial may be eligible for an additional open-label extension (OLE) phase of 2 years.
1. Study 1: Phase 2, proof of concept (POC) 26 week randomized control period (RCP)
2. Study 2: Phase 3, 52 week RCP

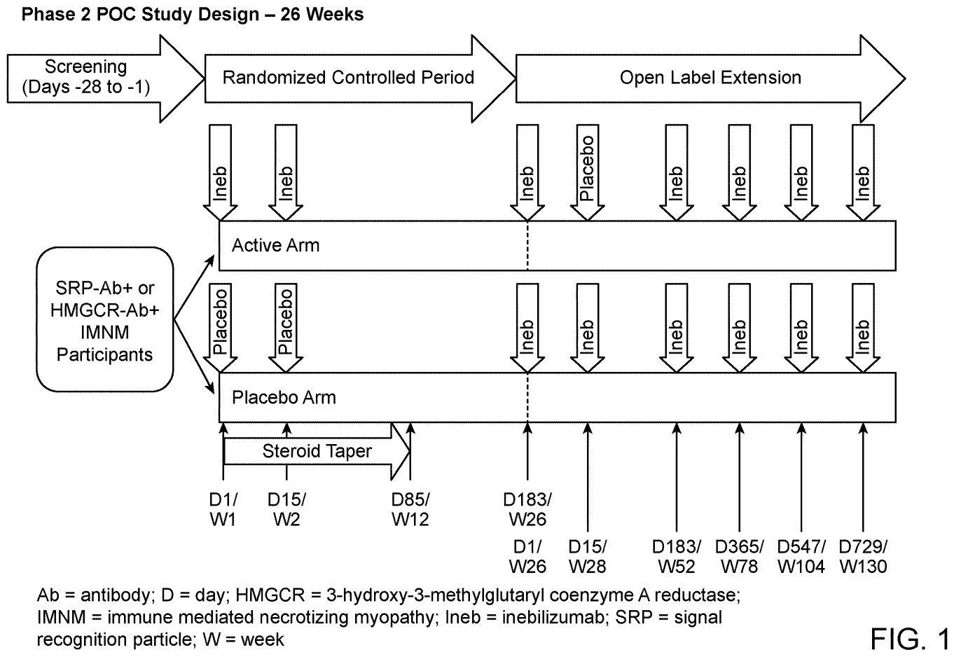

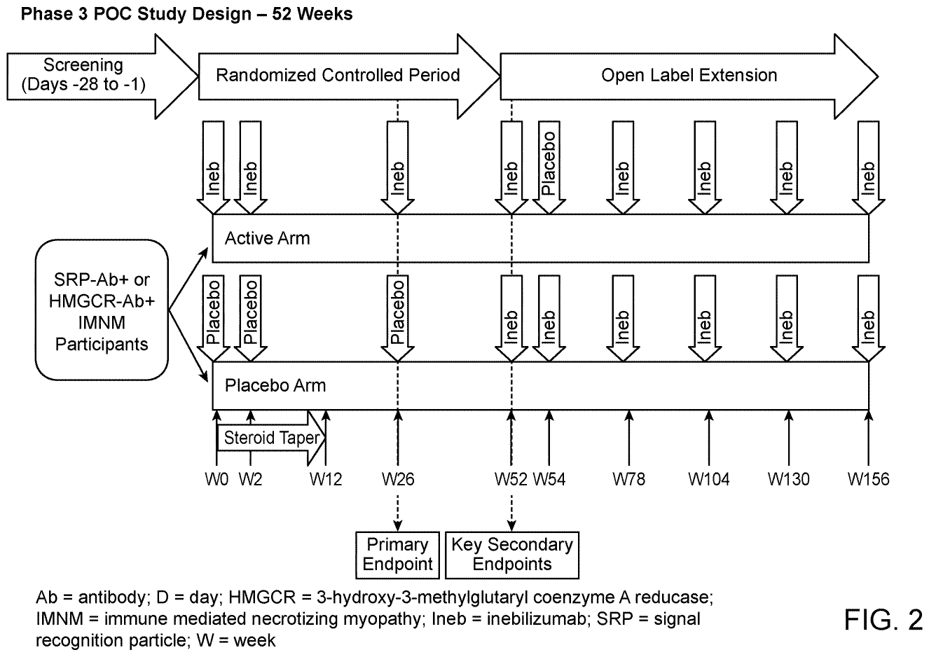

Approximately 170 adult subjects (40 in Phase 2 study and 130 in Phase 3 study) are enrolled. Both pools of subjects will be randomized 1:1 to receive either inebilizumab or placebo. Subjects will be stratified by their autoantibody type (SRP-Ab+ versus HMGCR-Ab+) to minimize the risk of baseline imbalances across treatment arms. For stage I, a minimum of 10 SRP-Ab+ and 10 HMGCR-Ab+ participants will be enrolled into each strata. For stage II, a minimum of 20 SRP-Ab+ and 20 HMGCR-Ab+ participants will be enrolled into each strata. An overview of the trial designs are presented in FIG. 1 and FIG. 2, and details of trial activities are provided in the schedule of assessments (Table 2) as well in Table 3 and Table 4.

TABLE 2
Exemplary schedule of assessments

| Study Period | Screening |
|---|---|
| Trial Visit | SV |
| Day of Study | D-28 to D-1 |
| Written informed consent | X |
| Review eligibility criteria | X |
| Demographics | X |
| Medical and disease history | X |
| Weight and height | X |
| Vital signs¹ | X |
| ECG | X |
| Concomitant medications | X |
| Physical exam (full)² | X |
| Collect urine for: | |
| Urinalysis | X |
| Collect blood for: | |
| Hematology and chemistry³ | X |
| Serum β-hCG⁴ | X |
| FSH⁵ | X |
| Serum virology⁶ | X |
| TB test (QuantiFERON ®-TB Gold) | X |
| Whole blood for flow cytometry (CD19+ B cell | X |
| count) | |
| SRP or HMGCR Ab serostatus assay | X |
| Total Ig, IgM, IgG, IgA | X |
| IMNM assessments | |
| Total MMT⁷ | X |
| PFT | X |

Ab = Antibody; β-hCG = beta human chorionic gonadotropin; D = day; ECG = electrocardiogram; FSH = follicle stimulating hormone; HMGCR = 3-hydroxy-3-methylglutaryl-CoA reductase; Ig = immunoglobulin; MMT = manual muscle testing; PFT = pulmonary function test; SRP = signal recognition particle; SV = screening visit; TB = tuberculosis.
¹Vital sign measurements include blood pressure, heart rate, respiratory rate, temperature.
²A full physical examination may be performed at screening, to include assessment of head, ears, eyes, nose, throat, lungs, heart, abdomen, skin, and extremities.
³Routine hematology and chemistry testing may include creatine kinase.
⁴For women of childbearing potential only as defined in the study inclusion criteria.
⁵For post-menopausal women only to confirm postmenopausal status.
⁶May include human immunodeficiency virus testing, hepatitis B testing (hepatitis B surface antigen and hepatitis core antibody [HBcAb] with reflex DNA testing), and hepatitis C virus antibody testing.
⁷MMT-8 may be used as study inclusion criteria.

TABLE 3
Exemplary proof of concept phase 2 study, randomized controlled period

| | Study Period | | | | | | | | |
|---|---|---|---|---|---|---|---|---|---|
| | Randomized Control Period | | | | | | | SFU | EDV²/CDV³ |
| | Trial Visit | | | | | | | | |
| | V1 | V2 | V3 | V4 | V5 | V6 | V7 | V15 | |
| | Week | | | | | | | | |
| | W 0 | W 2 | W 6 | W 12 | W 18 | W 22 | W 26 | W 142 | N/A |
| | RCP Day of Study | | | | | | | | |
| | D 1 | D 15 | D 43 | D 85 | D 127 | D 155 | D 183 | | N/A |
| | OLE Day of Study | | | | | | | | |
| | | | | | | | D 1 | D 813 | N/A |
| | Visit Window (±days) | | | | | | | | |
| | | ±3 | ±7 | ±7 | ±7 | ±7 | ±3 | ±7 | |
| Review eligibility criteria | X | | | | | | | | |
| Randomization⁴ | X | | | | | | | | |
| Weight | X | | | | | | X | X | X |
| Vital signs⁵ | X | X | X | X | X | X | X | X | X |
| ECG | X | X | | | | | X | X | X |
| Physical exam (abbreviated) | X | | | X | | | X | X | X |
| IMNM assessments | | | | | | | | | |
| 2016 ACR/EULAR TIS⁸ | X | X | X | X | X | X | X | | X |
| PFT | X | X | X | X | X | X | X | | X |
| Timed up and go test | X | | | X | | | X | | X |
| 2-minute walk test | X | | | X | | | X | | X |
| Short Form 12 or 36 | X | | | X | | | X | | X |
| PROMIS short form 7a (fatigue) | X | | | X | | | X | | X |
| PROMIS short form 8a (pain | X | | | X | | | X | | X |
| interference) | | | | X | | | | | |
| PROMIS short form 8b (physical | X | | | X | | | X | | X |
| function) | | | | | | | | | |
| Handheld dynamometry | X | | | X | | | X | | X |
| PGIS | X | | | X | | | X | | X |
| PGIC | X | | | X | | | X | | X |
| CGIS | X | | | X | | | X | | X |
| CGIC | X | | | X | | | X | | X |
| Myositis Functional Index | X | | | X | | | X | | X |
| Pain NRS | X | | | X | | | X | | X |
| EQ-5D-5L | X | | | X | | | X | | X |
| EQ-5D-5L-VAS | X | | | X | | | X | | X |
| Healthcare resource utilization | X | | | X | | | X | | X |
| Collect urine for: | | | | | | | | | |
| Urinalysis | X | X | X | X | X | X | X | X | |
| Pregnancy test⁶ | X | X | | | | | X | X | |
| Collect blood for: | | | | | | | | | |
| Hematology and chemistry | X | X | X | X | X | X | X | X | X |
| Creatine kinase | X | X | X | X | X | X | X | X | X |
| SRP or HMGCR Ab titer | X | | X | X | | | X | X | X |
| Total Ig, IgM, IgG, IgA | X | | | X | | | X | X | X |
| Inebilizumab PK | X⁷ | X⁷ | X | X | X | | X⁷ | | X |
| Inebilizumab ADA | X | | X | X | | | X | X | X |
| Whole blood for flow cytometry | X | X | X | X | | X | X | X | X |
| (CD20+ cell count; B & T cell | | | | | | | | | |
| subsets) | | | | | | | | | |
| Whole blood for PBMC isolation | X | | | X | | | X | X | X |
| Serum/plasma biomarkers | X | X | X | X | | X | X | X | X |
| RNA for transcriptomics | X | | X | X | | | X | X | X |
| Whole blood for DNA, SNP, and | X | | X | X | | | X | X | X |
| epigenetic analyses | | | | | | | | | |
| Substudies | | | | | | | | | |
| MRI | X | | | | | | X | | |
| Muscle biopsy | X | | | | | | X | | |

Ab = Antibody; ACR/EULAR = American College of Rheumatology/European League Against Rheumatism; ADA = anti-drug antibody; CDV = clinical deterioration visit; CGIS = Clinical Global Impression of Severity; CGIC = Clinical Global Impression of Change D = day; DNA = deoxyribonucleic acid; ECG = electrocardiogram; EDV = early discontinuation visit; HMGCR = 3-hydroxy-3-methylglutaryl-CoA reductase; Ig = immunoglobulin; IP = investigational product; MRI = magnetic resonance imaging; N/A = not applicable; OLE = open label extension; PBMC = peripheral blood mononuclear cells; PK = pharmacokinetics; PGIS= Patient Global Impression of Severity; PGIC = Patient Global Impression of Change; PROMIS = patient reported outcome measurement information system; RCP = randomized controlled period; RNA = ribonucleic acid; SNP = single nucleotide polymorphism; SRP = signal recognition particle; TIS = total improvement score; V = Visit; W = week.
All blood collections and assessments may be performed prior to drug administration on dosing days.
Footnotes:
1. All participants will return to the clinic 3 months (±7 days) following the Week 130 Visit of the OLP for a safety follow up visit.
²If a participant prematurely discontinues investigational product during the randomized controlled period or the open-label extension, they will return to the clinic and undergo an early discontinuation visit.
³If a participant experiences clinical deterioration during the randomized controlled period, they will come into the clinic and undergo a clinical deterioration visit.
⁴On Day 1, participants will be randomized in a 1:1 ratio to receive IV either: a) inebilizumab (300 mg) or b) placebo; baseline assessments may be performed prior to dosing.
⁵Vital signs (blood pressure, heart rate, respiratory rate, temperature) may be measured at all clinic visits.
⁶For women of childbearing potential as defined in the study inclusion criteria.
⁷Blood samples for pharmacokinetics may be drawn pre-dose and 15 (±5) minutes post-dose.
⁸Total MMT will be collected.

TABLE 4
Exemplary phase 3 study, randomized controlled period

| | Study Period | | | | | | | | | | | | | |
|---|---|---|---|---|---|---|---|---|---|---|---|---|---|---|
| | Randomized Control Period (RCP) | | | | | | | | | | | | | |
| | Trial Visit | | | | | | | | | | | | | |
| | | | | | | | | | | | | | | EDV¹⁰/ |
| | V1 | V2 | V3 | V4 | V5 | V6 | V7 | V8 | V9 | V10 | V11 | V12 | V13 | CDV¹¹ |
| | Week | | | | | | | | | | | | | |
| | | | | | | | | | | | | | W 52 or | |
| | W 0 | W 2 | W 6 | W 12 | W 18 | W 22 | W 26 | W 30 | W 34 | W 38 | W 42 | W 46 | EDV¹⁰ | N/A |
| | RCP Day of Study | | | | | | | | | | | | | |
| | D 1 | D 15 | D 43 | D 85 | D 127 | D 155 | D 183 | | | | | | | N/A |
| | Visit Window (±days) | | | | | | | | | | | | | |
| | | ±3 | ±7 | ±7 | ±7 | ±7 | ±3 | ±3 | ±7 | ±7 | ±7 | ±7 | ±7 | |
| Review eligibility criteria | X | | | | | | | | | | | | | |
| Randomization⁴ | X | | | | | | | | | | | | | |
| Weight | X | | | | | | X | | | | | | | X |
| Vital signs⁵ | X | X | X | X | X | X | X | X | X | X | X | X | X | X |
| ECG | X | X | | | | | X | X | | | | | | X |
| Physical exam | X | | | X | | | X | | | X | | | | X |
| (abbreviated) | | | | | | | | | | | | | | |
| IMNM assessments | | | | | | | | | | | | | | |
| 2016 ACR/EULAR TIS⁸ | X | X | X | X | X | X | X | X | X | X | X | X | X | X |
| PFT | X | X | X | X | X | X | X | X | X | X | X | X | X | X |
| Timed up and go test | X | | | X | | | X | | X | | | X | | X |
| 2-minute walk test | X | | | X | | | X | | X | | | X | | X |
| Short form 12 | X | | | X | | | X | | X | | | X | | X |
| PROMIS short form 7a | X | | | X | | | X | | X | | | X | | X |
| (fatigue) | | | | | | | | | | | | | | |
| PROMIS short form 8a | X | | | X | | | X | | X | | | X | | X |
| (pain) | | | | | | | | | | | | | | |
| PROMIS short form 8b | X | | | X | | | X | | X | | | X | | X |
| (physical function) | | | | | | | | | | | | | | |
| Handheld dynamometry | X | | | X | | | X | | X | | | X | | X |
| PGIS | X | | | X | | | X | | X | | | X | | X |
| PGIC | X | | | X | | | X | | X | | | X | | X |
| CGIS | X | | | X | | | X | | X | | | X | | X |
| CGIC | X | | | X | | | X | | X | | | X | | X |
| Myositis Functional Index | X | | | X | | | X | | X | | | X | | X |
| Pain NRS | X | | | X | | | X | | X | | | X | | X |
| EQ-5D-5L | X | | | X | | | X | | X | | | X | | X |
| EQ-5D-5L VAS | X | | | X | | | X | | X | X | | X | | X |
| Collect urine for: | | | | | | | | | | | | | | |
| Urinalysis | X | X | X | | | X | X | X | | | | | | |
| Pregnancy test⁶ | X | X | | | | | X | X | | X | | X | X | |
| Collect blood for: | | | | | | | | | | | | | | |
| Hematology and | X | X | X | X | X | X | X | X | X | X | X | X | X | X |
| chemistry | | | | | | | | | | | | | | |
| Creatine kinase | X | X | X | X | X | X | X | X | X | X | X | X | X | X |
| SRP or HMGCR Ab titer | X | | X | X | | | X | | X | X | | X | X | X |
| Total Ig, IgM, IgG, IgA | X | | | X | | | X | | | X | | X | | X |
| Inebilizumab PK | X⁷ | X⁷ | X | X | X | | X⁷ | X⁷ | X | X⁷ | | | X⁷ | X |
| Inebilizumab ADA | X | | X | X | | | X | | X | X | | X | X | X |
| Whole blood for flow | X | X | X | X | | X | X | X | X | X | | X | X | X |
| cytometry (CD20+ cell | | | | | | | | | | | | | | |
| count; B & T cell | | | | | | | | | | | | | | |
| subsets) | | | | | | | | | | | | | | |
| Whole blood for PBMC | X | | | X | | | X | | X | X | | X | X | X |
| isolation | | | | | | | | | | | | | | |
| Serum/plasma | X | X | X | X | | X | X | X | X | X | | X | X | X |
| biomarkers | | | | | | | | | | | | | | |
| RNA for transcriptomics | X | | X | X | | | X | X | X | X | | X | X | X |
| Whole blood for DNA, | X | | X | X | | | X | | | X | | X | X | X |
| SNP, and epigenetic | | | | | | | | | | | | | | |
| analyses (optional) | | | | | | | | | | | | | | |
| Substudies: | | | | | | | | | | | | | | |
| MRI (optional) | X | | | | | | X | | | | | | | |
| Muscle biopsy (optional) | X | | | | | | X | | | | | | | |
| IP administration | X | X | | | | | X | | | | | | X | |
| Concomitant medications | X | X | X | X | X | X | X | X | X | X | X | X | X | X |

Ab = Antibody; ACR/EULAR = American College of Rheumatology/European League Against Rheumatism; ADA = anti-drug antibody; AE = adverse event; CGIS = Clinical Global Impression of Severity CGIC = Clinical Global Impression of Change CDV = clinical deterioration visit; D = day; DNA = deoxyribonucleic acid; ECG = electrocardiogram; EDV = early discontinuation visit; HMGCR = 3-hydroxy-3-methylglutaryl-CoA reductase; Ig = immunoglobulin; IP = investigational product; MRI = magnetic resonance imaging; N/A = not applicable; OLE = open label extension; PBMC = peripheral blood mononuclear cells; PGIS = Physician Global Impression of Severity; PGIC = Physician Global Impression of Change; PK = pharmacokinetics; PROMIS = patient reported outcome measurement information system; RCP = randomized controlled period; RNA = ribonucleic acid; SNP = single nucleotide polymorphism; SRP = signal recognition particle; TIS = total improvement score; V = Visit; W = week.
Blood collections and assessments may be performed prior to drug administration on dosing days.
Footnotes:
9. All participants may return to the clinic 3 months (±7 days) following the Week 130 Visit of the OLP for a safety follow up visit.
¹⁰If a participant prematurely discontinues investigational product during the randomized controlled period or the open-label extension, they will return to the clinic and undergo an early discontinuation visit.
¹¹If a participant experiences clinical deterioration during the randomized controlled period, they will come into the clinic and undergo a clinical deterioration visit.
12. On Day 1, participants will be randomized in a 1:1 ratio to receive IV either: a) inebilizumab (300 mg) or b) placebo; baseline assessments will be performed prior to dosing.
13. Vital signs (blood pressure, heart rate, respiratory rate, temperature) will be measured at all clinic visits.
14. For women of childbearing potential as defined in the study inclusion criteria.
15. Blood samples for pharmacokinetics will be drawn pre-dose and 15 (±5) minutes post-dose.
16. All TIS assessments should be conducted by the same assessor if possible. Total MMT will be collected.

In the Phase 2 stage, participants entering the study on corticosteroids will remain on a stable dose of corticosteroids throughout the treatment period. In the Phase 3 stage, participants will follow the protocol-defined corticosteroid taper outlined below (Table 5). In the Phase 3 trial, no steroid taper should occur until after the Week 26 timepoint.

TABLE 5
Exemplary corticosteroid tapering schedule
post week 26 during the phase 3

| RCP Week | Oral Prednisone Dose (or Equivalent) | |
|---|---|---|
| Weeks 26-27 | 30 | mg |
| Weeks 28-29 | 25 | mg |
| Weeks 29-30 | 20 | mg |
| Weeks 31-32 | 15 | mg |
| Weeks 33-34 | 10 | mg |
| Weeks 35-36 | 7.5 | mg |

Subjects entering the Phase 3 study on corticosteroids may begin a per protocol corticosteroid taper following Week 26 of the RCP with the goal of all participants reaching either 7.5 mg/day or a 50% reduction in corticosteroid dose on or before Week 35 (Table 5). Participants will maintain this stable dose through the end of the RCP.

### Clinical Deterioration (Both Studies)

Participants may be evaluated for a clinical deterioration visit if clinical worsening occurs. Protocol defined clinical deterioration includes the following criteria:
1. In the investigator's opinion, the participants' health is in jeopardy, which is supported by creatine kinase (CK) elevation or a significant worsening on total improvement score (TIS) assessments as defined below:
   a. A doubling of baseline CK levels
   b. A 20-point worsening in TIS

All subjects who are planned to receive rescue therapy must meet criteria 1 plus either (a) or (b). Additionally, the sponsor should be contacted on an urgent basis should a protocol-defined clinical deterioration be identified.

### Study Population

The following eligibility criteria apply to both the Phase 2 and the Phase 3 studies.

### Inclusion Criteria

Participants are eligible to be included in the studying if the following criteria are met:
1. Adults and adolescents ≥16 years old and <80 years old.
2. Historical diagnosis of IMNM defined as a positive serologic test for anti-SRP or anti-HMGCR antibody titers within the past 5 years. This will be confirmed centrally.
3. Clinical evidence of active disease defined as achieving both:
   a. CK>1000 U/L
   b. Score of ≤136 units on MMT-8
4. Subjects may enter the study on the following baseline medications:
   a. No disease-specific treatment
   b. Prednisone or equivalent (≤20 (or 15) mg/day) with stable dosing for a minimum of 2 weeks prior to screening with continued stability prior to randomization
   c. Non-steroidal ISTs with a minimum duration of 12 weeks of treatment with stable dosing for a minimum of 8 weeks prior to screening and continued stability prior to randomization:
      i. Methotrexate
      ii. Azathioprine
      iii. Mycophenolate mofetil
      iv. Mycophenolic acid
      v. Tacrolimus
      vi. Cyclosporine
5. Females of childbearing potential who are sexually active with a non-sterilized male partner must use at least one highly effective contraception method (Table 6) from the time of screening and for 6 months after the final dose of investigational product (IP). Periodic abstinence, the rhythm method, and the withdrawal method are not acceptable methods of contraception.
   a. Note: Females of childbearing potential are defined as those who are not surgically sterile (i.e., bilateral tubal ligation, bilateral oophorectomy, or complete hysterectomy) or those who are not postmenopausal (defined as 12 months with no menses without an alternative medical cause and a follicle-stimulating hormone [FSH] level in the postmenopausal range [≥16.70 mIU/mL]). If the FSH level is not in the postmenopausal range in a subject with amenorrhea, she may still enroll in the study but must follow the same contraception requirements as women of childbearing potential.

TABLE 6
Exemplary methods of highly effective contraception

| Barrier Methods | Hormonal Methods |
|---|---|
| Intrauterine device | Combined oral (estrogen and progestogen |
| Intrauterine hormone-releasing system ^{a} | containing hormonal contraception) |
| Bilateral tubal occlusion | Injectable |
| Vasectomized partner ^{b} | Transdermal (patch) |
| Sexual abstinence ^{c} | Progestogen-only hormonal contraception |
| | associated with inhibition of ovulation ^{d} |
| | Implantable |
| | Intravaginal |

^{a} This is also considered a hormonal method.
^{b} With appropriate post-vasectomy documentation of surgical success (absence of sperm in ejaculate).
^{c} Sexual abstinence is considered a highly effective method only if defined as refraining from heterosexual intercourse during the entire period of the study and if it is the preferred and usual lifestyle of the subject.
^{d} Progestogen-only hormonal contraception, where inhibition of ovulation is not the primary mode of action (e.g., minipill), is not accepted as a highly effective method.

6. Non-sterilized males who are sexually active with a female partner of childbearing potential must use a condom from Day 1 for the duration of the study and for 3 months after the last dose of investigational product (IP). Because male condom is not a highly effective contraception method, it is strongly recommended that female partners of a male study subject also use a highly effective method of contraception throughout this period (Table 6).

### Exclusion Criteria

Participants will be ineligible for trial participation if they meet any of the following criteria:

### General Exclusion Criteria

1. Any condition that, in the opinion of the Investigator, would place the participant at unacceptable risk of complications, interfere with evaluation of the IP, or confound the interpretation of participant safety or study results.
2. Lactating or pregnant females, or females who intend to become pregnant anytime from signing the informed consent form (ICF) throughout the RCP plus 6 months following last dose of IP.
3. Spontaneous or induced abortion, still or live birth≤4 weeks prior to screening.
4. Body weight <37.5 kg.
5. History of drug or alcohol abuse within <1 year prior to screening, or any condition associated with poor compliance as judged by the Investigator.
6. History of severe allergic or anaphylactic reactions to biologic agents or known allergy to any component of the IP formulation.
7. History of recurrent significant infections (e.g., requiring hospitalization or intravenous [IV] antibiotics).
8. Within 2 weeks prior to the screening visit: clinically significant active infection requiring antimicrobial medication but allowing chronic nail infections.
9. History of cancer, except for the following:
   a. In situ carcinoma of the cervix treated with apparent success with curative therapy for >12 months prior to screening.
   b. Cutaneous basal cell or squamous cell carcinoma treated with apparent success with curative therapy for >12 months prior to screening.
   c. Prostate cancer treated with radical prostatectomy or radiation therapy with curative intent >3 years prior to screening and without known recurrence or current treatment.
10. Any of the following laboratory abnormalities at screening (one repeat test may be conducted to confirm results prior to randomization within the same screening period):
   a. Elevated liver enzymes (aspartate aminotransferase (AST) or alanine aminotransferase (ALT)>2.5×upper limit of normal (ULN) unless attributable to IMNM underlying disease with investigator and medical monitor approval)
   b. Total bilirubin >1.5×ULN (unless due to Gilbert's syndrome)
   c. Estimated glomerular filtration rate (eGFR)<45 mL/min/1.73 m²
   d. CD19+ B cell count <40 cells/μL
   e. Absolute neutrophil count (ANC)<1.2×10³ cells/μL
   f. Platelet count <75,000/μL (or <75× 109/L)
   g. Hemoglobin <8.0 g/dL
   h. Total Ig<600 mg/dL
11. Known immunodeficiency disorder, including current infection or positive test for human immunodeficiency virus (HIV).
12. Positive test for chronic hepatitis B infection at screening, defined as either (1) positive hepatitis B surface antigen (HBsAg) or (2) a positive hepatitis B core antibody (anti-HBc) PLUS negative hepatitis B surface antibody (anti-HBs).
   a. Note: Subjects with a positive anti-HBs only, or a positive anti-HBc plus positive anti-HBs and negative HBsAg, are eligible to enroll.
13. Positive test for hepatitis C virus antibody.
14. History of active or latent tuberculosis (TB), or a positive QuantiFERON®-TB Gold test at screening, unless treatment for tuberculosis was completed per local guidelines. Subjects with latent TB or a positive QuantiFERON®-TB Gold test who are actively on anti-TB treatment can enroll if they have completed at least 1 month of anti-TB treatment and intend to complete the full course of anti-TB treatment. Subjects with an indeterminate QuantiFERON®-TB Gold test result can enroll if a repeat QuantiFERON®-TB Gold is negative or a tuberculin skin test is negative.

### Disease-Related Exclusion Criteria

15. Statin medication use within 28 days prior to screening or anticipated use during the study (if HMGCR-Ab+).
16. Currently wheelchair bound or routine wheelchair use.
17. Uncontrolled or rapidly progressive ILD.
18. History of clinically significant cardiac disease.

### Prior and Concomitant Therapy Exclusion Criteria

19. Concurrent/previous enrollment in another clinical study involving receipt of an investigational treatment within 4 weeks or 5 half-lives of the investigational treatment, whichever is longer, prior to screening.
20. Receipt of any biologic B cell-depleting therapy (e.g., rituximab, ocrelizumab, obinutuzumab, ofatumumab, inebilizumab) or any experimental B cell depleting agent in the 6 months prior to screening.
21. Receipt of any other monoclonal antibody (mAb) or large molecule biologic, including but not limited to FcRn inhibitors, anti-TNF mAbs, anti-JAK Stat mAbs, and complement inhibitors within 3 months or 5 half-lives of the treatment, whichever is longer, prior to screening.
22. Receipt within the 4 weeks prior to screening:
   a. IVIg
   b. Plasma exchange (PLEX) treatment.
   c. Live attenuated vaccine (administration of inactivated [killed] vaccine is acceptable).
   d. Blood transfusion.

### Prohibited Medications

The following medications and treatments will not be permitted during the study:
1. Live attenuated vaccines. Commonly used live vaccines include the Zostavax® herpes zoster (shingles) vaccine; intranasal flu vaccine; measles, mumps, rubella vaccine; rotavirus vaccine; typhoid oral vaccine; yellow fever vaccine; smallpox vaccine; and adenovirus vaccine.
2. IVIg or plasma exchange is limited to rescue therapy for protocol defined clinical deterioration.
3. Any introduction or dose escalation of corticosteroids is limited to rescue therapy for protocol defined clinical deterioration.
4. Statin medication (if HMGCR-Ab+).
5. Any monoclonal antibody (mAb) or large molecule biologic, including but not limited to B cell depleting mAbs, FcRn inhibitors, anti-TNF mAbs, anti-JAK Stat mAbs, and complement inhibitors.
6. Other immunosuppressive or immunomodulatory medications, unless agreed upon following consultation with the sponsor medical monitor or already in use at the time of randomization.

### Dose Regiment and Route of Administration

For the treatment of IMNM, inebilizumab will be administered as a 300 mg intravenous (IV) infusion on Days 1 and 15 and then every 6 months thereafter.

In this study, participants will receive IV infusions of either inebilizumab (300 mg) or placebo at the following timepoints:
For the Phase 2 trial: on Day 1, Day 15, and Week 26.
For the Phase 3 trial: on Day 1, Day 15, Week 26, and Week 52

All participants entering the OLE will receive IV infusions of inebilizumab (300 mg) on OLE Day 1 and every 6 months thereafter for a period of 2 years. For Day 15 of the OLE participants originally assigned to inebilizumab arm of the RCP will receive placebo while those originally assigned to the placebo arm will receive inebilizumab 300 mg IV on Day 15 in order to avoid unblinding of the original participant treatment assignment.

Prior to all IP infusions, all participants may be premedicated with site-supplied IV methylprednisolone (100 mg or equivalent glucocorticoid), oral diphenhydramine (25-50 mg or equivalent antihistamine), and oral paracetamol (acetaminophen; 500-650 mg) prophylactically to reduce the risk of infusion reactions.

### Dosage Form and Strength Formulation

Inebilizumab is a sterile liquid dosage form (100 mg inebilizumab per vial, in 10 mL label-claim volume) intended for IV infusion following dilution with normal saline.

### Duration of Treatment and Follow-Up

### Phase 2

The planned duration of the double-blind randomized controlled period is 26 weeks. The planned duration of the open-label extension period is 2 years.

### Phase 3

The planned duration of the double-blind randomized controlled period is 52 weeks. The planned duration of the open-label extension period is 2 years.

### Eligibility for Open-Label Period (OLP)

Subjects eligible for the OLP must:
1. Have completed the RCP Week 26 (for the Phase 2 trial) or the RCP Week 52 visit Phase 3 trial).
2. Have completed a 28-day washout period after discontinuation of non-B cell-depleting, non-GC immunosuppressive therapy initiated during the RCP, if relevant.
3. Receive Dose 1 in the OLP within the window of 1-38 days after the RCP Week 26 (Phase 2) or Week 52 (Phase 3) visit (the final visit for the RCP and the first visit for the OLP cannot occur on the same day).
4. Have a planned taper of GCs to discontinuation by 8 weeks after OLP Dose 1, if receiving GC at the time of OLP entry.

To maintain the every-6-month dosing of IP, subjects may enroll in the OLP and be dosed as soon as is feasible after completion of the RCP.

Table 7 below summarizes the schedule of procedures for the OLP for both studies.

TABLE 7
Exemplary open-label assessments and procedures

| | Study Period | | | | | | | | | |
|---|---|---|---|---|---|---|---|---|---|---|
| | OLP Visit Number | | | | | | | | | |
| | | | | | | | V9, | | | |
| | V1 | V2 | V3, V4 | V5 | V6, V7 | V8 | V10 | V11 | SFU | EDV/CDV |
| | OLP Day | | | | | | | | | |
| | | Day | | Day | Day | Day | Day | Day | | |
| | Day 0 ^{a} | 15 | Day 85 | 183 | 267 | 365 | 547 | 729 | Day 813 | N/A |
| | Window | | | | | | | | | |
| | ±3 | ±3 | ±7 | ±7 | ±7 | ±7 | ±7 | ±7 | ±7 | N/A |
| | RCP Week | | | | | | | | | |
| | Wk. 26 | | | | | | | | | |
| | OR 52 | | | | | | | | | |
| General | | | | | | | | | | |
| Assessments/Procedures | | | | | | | | | | |
| Vital signs | X | X | X | X | X | X | X | X | X | X |
| Weight | X | | | | | | | X | X | X |
| Physical exam | X | | | X | | | | X | | X |
| (abbreviated) | | | | | | | | | | |
| ECG | X | X | | | | | | X | X | X |
| Safety | | | | | | | | | | |
| Assessment of AEs, | X | X | X | X | X | X | X | X | X | X |
| SAEs, AESIs | | | | | | | | | | |
| Concomitant medication | X | X | X | X | X | X | X | X | X | X |
| IMNM assessments | | | | | | | | | | |
| 2016 ACR/EULAR | X | X | X | X | X | X | X | X | | X |
| TIS⁸ | | | | | | | | | | |
| PFT | X | X | X | X | X | X | X | X | | X |
| Timed up and go test | X | | X | X | | X | | X | | X |
| 2-minute walk test | X | | X | X | | X | | X | | X |
| Short form 12 | X | | | X | | X | | X | | X |
| PROMIS short form | X | | | X | | X | | X | | X |
| 7a (fatigue) | | | | | | | | | | |
| PROMIS short form | X | | | X | | X | | X | | X |
| 8a (pain) | | | | | | | | | | |
| PROMIS short form 8b | | | | X | | X | | X | | X |
| (physical function) | | | | | | | | | | |
| Handheld dynamometry | X | | | X | | X | | X | | X |
| PGIS | X | | | X | | X | | X | | X |
| PGIC | X | | | X | | X | | X | | X |
| CGIS | X | | | X | | X | | X | | X |
| CGIC | X | | | X | | X | | X | | X |
| Myositis Functional | X | | | X | | X | | X | | X |
| Index | X | | | X | | X | | X | | X |
| Pain NRS | X | | | X | | X | | X | | X |
| EQ-5D-5L | X | | | X | | X | | X | | X |
| EQ-5D-5L VAS | X | | | X | | X | | X | | X |
| Collect urine for: | | | | | | | | | | |
| Urinalysis | X | X | X | X | X | X | X | X | X | X |
| Pregnancy test⁶ | X | X | | X | | X | X | X | X | X |
| Collect blood for: | | | | | | | | | | |
| Hematology and | X | X | X | X | X | X | X | X | X | X |
| chemistry | | | | | | | | | | |
| Creatine kinase | X | X | X | X | X | X | X | X | X | X |
| SRP or HMGCR Ab | X | | X | X | | X | X | X | X | X |
| titer | | | | | | | | | | |
| Total Ig, IgM, IgG, | X | | | X | | X | | X | X | X |
| IgA | | | | | | | | | | |
| Inebilizumab PK | X⁷ | X⁷ | X | X | | | X | X⁷ | | X⁷ |
| Inebilizumab ADA | X | | X | X | | X | X | X | X | X |
| Whole blood for flow | X | | X | X | | X | X | X | X | X |
| cytometry (CD20+ | | | | | | | | | | |
| cell count; B & T cell | | | | | | | | | | |
| subsets) | | | | | | | | | | |
| Whole blood for | X | | | | | | | | | |
| PBMC isolation | | | | | | | | | | |
| Serum/plasma | X | X | X | X | | X | X | X | X | X |
| biomarkers | | | | | | | | | | |
| RNA for | X | X | X | X | | X | X | X | X | X |
| transcriptomics | | | | | | | | | | |
| Whole blood for | X | | | X | | X | X | X | X | X |
| DNA, SNP, and | | | | | | | | | | |
| epigenetic analyses | | | | | | | | | | |
| (optional) | | | | | | | | | | |
| Assessment of eligibility | X | | | | | | | | | |
| for OLP | | | | | | | | | | |
| IP administration | X | X | | X | | X | X | X | | |

^{a} Day 0 of the OLP corresponds to Week 26 (for Phase 2) or Week 52 (Phase 3) of the respective RCP that the subject is assigned to.

### Criteria for Evaluation

The 2016 ACR/EULAR total improvement score (TIS) at RCP Week 26 will be used as the primary efficacy outcome measure. The TIS has been developed and validated for dermatomyositis (DM) and polymyositis (PM) by the ACR/EULAR Board of Directors (Aggarwal et al, 2017) and is derived from standardized clinical response criteria that are recommended for use as primary endpoint in myositis therapeutic trials. Improvement category thresholds for minimal, moderate, and major improvement have been developed and validated for DM and PM, but no specific thresholds have been developed for IMNM (Aggarwal et al, 2017). In the current design, a validation study for the TIS in IMNM will occur in conjunction with the Phase 2 study. Subjects who are participants in the validation study will not be participants in the Phase 2 study, although may be eligible for the Phase 3 study. Therefore, for the primary endpoint, the continuous rather than categorical outcome will be used. Continuous measures may also provide greater power to detect change and have greater sensitivity to change (Rider et al, 2018).

### Statistical Analyses

Analyses of the Phase 2 stage and Phase 3 stage will be conducted separately. This section summarizes the planned statistical analyses for both stages unless otherwise stated. Further details will be provided in a comprehensive statistical analysis plan (SAP).

### All Subject Analysis Set

This analysis set includes all subjects screened for the study and may be used for the reporting of disposition.

### Full Analysis Set

The full analysis set (FAS) includes all subjects randomized who received at least 1 dose of IP in the study. Subjects may be analyzed according to the treatment randomized. The efficacy analysis will be based on the FAS.

### Pharmacokinetic (PK) Analysis Set

The PK analysis set includes all subjects who receive IP and have at least 1 quantifiable serum PK observation post-first dose. Subjects will be analyzed according to the treatment that they actually receive. The PK analysis will be based on the PK analysis set.

### The Any Inebilizumab Analysis Set

The Any Inebilizumab Analysis Set will include all subjects who receive any dose of inebilizumab.

### INCORPORATION BY REFERENCE

This patent application incorporates by reference in their entireties for all purposes the following patent publications and applications: Int'l Appl. Nos. PCT/US2020/29613 and PCT/US2007/077916.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8.

2. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, and wherein the subject in need thereof is pediatric.

3. The method of claim 2, wherein the pediatric subject is 16-18 years old.

4. The method of any one of claims 1-3, wherein the subject is signal recognition particle-antibody positive (SRP-Ab+), 3-hydroxy-3-methylglutaryl-coenzyme A reductase-antibody positive (HMGCR-Ab+), or both SRP-Ab+ and HMGCR-Ab+.

5. The method of claim 4, wherein the subject is SRP-Ab+.

6. The method of claim 4, wherein the subject is HMGCR-Ab+.

7. The method of any one of claims 1-3, wherein the subject is signal recognition particle-antibody negative (SRP-Ab−), 3-hydroxy-3-methylglutaryl-coenzyme A reductase-antibody negative (HMGCR-Ab−), or both SRP-Ab− and HMGCR-Ab−.

8. The method of any one of claims 1-7, wherein the subject in need was previously treated with a conventional therapy.

9. The method of claim 8, wherein the conventional therapy comprises a corticosteroid, a non-steroidal immunosuppressant (IST), antibody therapy, or any combination thereof.

10. The method of claim 9, wherein the conventional therapy is a corticosteroid, and wherein the corticosteroid is prednisone.

11. The method of claim 9, wherein the conventional therapy is an IST selected from the group consisting of: methotrexate, cyclophosphamide, cyclosporine, azathioprine, mycophenolate mofetil, mycophenolic acid, tacrolimus, and any combination thereof.

12. The method of claim 9, wherein the conventional therapy is an antibody therapy is selected from the group consisting of: rituximab, ocrelizumab, obinutuzumab, ofatumumab, inebilizumab intravenous immunoglobulin (IVIg), and combinations thereof.

13. The method of any one of claims 8-12, wherein the conventional therapy is tapered following the administering of the effective amount of the anti-CD19 antibody or antigen-binding fragment thereof.

14. The method of claim 13, wherein the tapering comprises reducing a dosage of the conventional therapy every two weeks beginning on week 26.

15. The method of claim 13 or 14, wherein the conventional therapy comprises prednisone, and wherein the prednisone is tapered by 5 mg every two weeks following the administering.

16. The method of claim 15, wherein the tapering is continued at least until week 12.

17. The method of any one of claims 4-16, wherein the subject was determined to have at least one of SRP-Ab or HMGCR-Ab before the administering.

18. The method of any one of claims 1-17, wherein the administering of the anti-CD19 antibody or antigen-binding fragment thereof is repeated.

19. The method of any one of claims 1-17, wherein the administering is repeated on weeks 2, 26, 52, 78, 104, 130, and 156.

20. The method of any one of claims 1-19, wherein the administering, the repeated administering, or both are effective at reducing disease or symptoms thereof by week 12, 26, or 52.

21. The method of any one of claims 1-19, wherein the administering, the repeated administering, or both are effective at reducing disease or symptoms thereof by week 26 following the administering as determined by an increased 2016 ACR/EULAR total improvement score (TIS) as compared to a baseline score of the subject or a score in an untreated subject.

22. The method of any one of claims 1-19, wherein the administering, the repeated administering, or both are effective at reducing disease or symptoms thereof by week 26 following the administering as compared to a baseline level of the subject or a level in an untreated subject.

23. The method of claim 22, wherein the reduced disease or symptoms thereof are determined by an improvement in muscle weakness, reduction in serum CK level, reduced atrophy, reduced edema, reduced fatty tissue replacement, reduced interstitial lung disease, reduced B cell levels, MMT8 score, and combinations thereof.

24. The method of any one of claims 1-23, wherein the subject is human.

25. The method of any one of claims 21-23, wherein the disease reduction is of at least about 3-fold, 5-fold, 20-fold, 40-fold, 60-fold, 80-fold, or up to about 100-fold.

26. The method of any one of claims 21-23, wherein the TIS score is ≥5, 10, 20, 30, 40, 50, 60, 80, or 100.

27. The method of any one of claims 1-26, wherein before the administering the subject in need thereof is prophylactically administered a glucocorticoid, an antihistamine, an anti-pain agent, and any combination thereof.

28. The method of claim 27, wherein the subject is prophylactically administered the glucocorticoid, the antihistamine, and the anti-pain agent.

29. The method of any one of claims 1-26, wherein the subject is prophylactically administered methylprednisolone, diphenhydramine, and acetaminophen.

30. The method of any one of claims 1-29, wherein the effective amount of the anti-CD19 antibody or antigen-binding fragment thereof is from about: 50 mg to about 1000 mg, 100-800 mg, 200-600 mg, 200-500 mg, 250-450 mg, 200-350 mg, 250-350 mg, or 375-325 mg.

31. The method of any one of claims 1-29, wherein the effective amount is from about: 50 mg, 100 mg, 200 mg, 225 mg, 250 mg, 275 mg, 300 mg, 325 mg, 350 mg, 375 mg, 400 mg, 500 mg, 600 mg, 700 mg, 800 mg, 900 mg, or 1000 mg.

32. The method of claim 31, wherein the effective amount is about 300 mg.

33. The method of any one of claims 1-32, wherein the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively.

34. The method of any one of claims 1-32, wherein the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.

35. The method of any one of claims 1-34, wherein the anti-CD19 antibody is a monoclonal antibody.

36. The method of any one of claims 1-35, wherein the anti-CD19 antibody is afucosylated.

37. The method of any one of claims 18-36, wherein the administering, the repeated administering, or both are effective at eliminating the IMNM for at least about 3 months, 6 months, 1 year, or 2 years.

38. The method of any one of claims 18-37, wherein the administering, the repeated administering, or both are effective at stabilizing or reducing the IMNM for at least about 3 months, 6 months, 1 year, or 2 years.

39. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is from about 100 mg-1000 mg.

40. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is about 300 mg.

41. A method of treatment, the method comprising administering an effective amount of an anti-CD19 antibody or antigen-binding fragment thereof to a pediatric subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively.

42. A method of treating Immune Mediated Necrotizing Myopathy (IMNM), the method comprising administering an effective amount of inebilizumab to a subject in need thereof, wherein the effective amount is about 100 mg-1000 mg.

43. A method of treatment, the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is positive for autoantibodies selected from the group consisting of: signal recognition particle (SRP), 3-hydroxy-3-methylglutaryl-coenzyme A reductase (HMGCR), and both SRP and HMGCR.

44. A method of treatment, the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, and wherein the subject is negative for autoantibodies selected from the group consisting of: signal recognition particle (SRP), 3-hydroxy-3-methylglutaryl-coenzyme A reductase (HMGCR), and both SRP and HMGCR.

45. The method of any one of claims 1-44, wherein the administering comprises 300 mg of the anti-CD19 antibody or antigen-binding fragment thereof on day 1 and day 15, followed by a single 300 mg infusion approximately every 6 months thereafter.

46. A method of treating Immune Mediated Necrotizing Myopathy (IMNM), the method comprising administering an effective amount of an anti-CD19 antibody or antigen-binding fragment thereof to a subject in need thereof, wherein the anti-CD19 antibody or antigen-binding fragment thereof comprises Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 2, 3, 4, 6, 7, and 8, respectively, wherein the effective amount is about 300 mg and is administered every 6 months, and wherein two weeks prior to the first 300 mg administration every 6 months, an initial 300 mg dose is administered.

47. The method of any one of claims 38-46, wherein the anti-CD19 antibody comprises a heavy chain variable region (VH) and a light chain variable region (VL), wherein the VH and the VL regions comprise an amino acid sequence at least 80% identical to SEQ ID NO: 1 and SEQ ID NO: 5, respectively.

48. The method of any one of claims 38-46, wherein the anti-CD19 antibody comprises a heavy chain variable region (VH) and a light chain variable region (VL), wherein the VH and the VL regions comprise the amino acid sequence of SEQ ID NO: 1 and SEQ ID NO: 5, respectively.
